(19) United States
(12) Patent Application Publication
(10) Pub. No.: US 2003/0161867 A1
(43) Pub. Date: Aug. 28, 2003

(54) Skin-permeable selective cyclooxygenase-2 inhibitor composition

(76) Inventors: Guang Wei Lu, Ann Arbor, MI (US); Gary D. Ewing, Kalamazoo, MI (US); Praveen Tyle, Kalamazoo, MI (US); Brenda M. Stoller, Portage, MI (US); Rajeev Gokhale, Libertyville, IL (US); Ashwini Gadre, St. Louis, MO (US)

Correspondence Address: Pharmacia Corporation, Corporate Patent Department, 800 N. Lindbergh Boulevard -04B, St. Louis, MO, 63167, US

(21) Appl. No.: 10/158,342

(22) Filed: May 30, 2002

Related U.S. Application Data
(60) Provisional application No. 60/294,838, filed on May 31, 2001.
(60) Provisional application No. 60/350,756, filed on Nov. 13, 2001.

(51) Int. Cl. A61K 31/50; A61K 31/415; A61K 9/70; A61K 31/365; A61K 31/195; A61K 31/12

(52) U.S. Cl. 424/449; 514/247; 514/406; 514/471; 514/567; 514/684

(57) ABSTRACT

A dermally deliverable pharmaceutical composition comprises at least one selective cyclooxygenase-2 (COX-2) inhibitory drug or prodrug thereof solubilized in a pharmaceutically acceptable carrier that comprises a low molecular weight monohydric alcohol, and exhibits a skin permeation rate of the therapeutic agent at least equal to that exhibited by a reference solution of the therapeutic agent in 70% aqueous ethanol. A method of effecting targeted delivery of a selective COX-2 inhibitory drug to a site of pain and/or inflammation in a subject comprises topically administering such a composition to skin of the subject, preferably at a locus overlying or adjacent to the site of pain and/or inflammation. A method of effecting systemic treatment of a subject having a COX-2 mediated disorder comprises transdermally administering such a composition, preferably by contacting the composition with an area of skin of the subject not greater than about 400 cm².

This application claims priority of U.S. provisional application Serial No. 60/294,838, filed on May 31, 2001, and U.S. provisional application Serial No. 60/350,756, filed on Nov. 13, 2001.

### FIELD OF THE INVENTION

The present invention relates to pharmaceutical compositions containing a selective cyclooxygenase-2 (COX-2) inhibitory drug, in particular to such compositions that are suitable for administration to skin to provide a local or systemic therapeutic effect. The invention also relates to processes for preparing such compositions and to methods of treatment comprising administration of such compositions to skin of a subject in need thereof.

### BACKGROUND OF THE INVENTION

Inhibition of cyclooxygenase (COX) enzymes is believed to be at least the primary mechanism by which nonsteroidal anti-inflammatory drugs (NSAIDs) exert their characteristic anti-inflammatory, antipyretic and analgesic effects, through inhibition of prostaglandin synthesis. Conventional NSAIDs such as ketorolac, diclofenac, naproxen and salts thereof inhibit both the constitutively expressed COX-1 and the inflammation-associated or inducible COX-2 isoforms of cyclooxygenase at therapeutic doses. Inhibition of COX-1, which produces prostaglandins that are necessary for normal cell function, appears to account for certain adverse side effects that have been associated with use of conventional NSAIDs. By contrast, selective inhibition of COX-2 without substantial inhibition of COX-1 leads to anti-inflammatory, antipyretic, analgesic and other useful therapeutic effects while minimizing or eliminating such adverse side effects. Selective COX-2 inhibitory drugs have therefore represented a major advance in the art.

Numerous compounds have been reported having therapeutically and/or prophylactically useful selective COX-2 inhibitory effect, and have been disclosed as having utility in treatment or prevention of specific COX-2 mediated disorders or of such disorders in general. Among such compounds are a large number of substituted pyrazolyl benzenesulfonamides as reported in U.S. Pat. No. 5,466,823 to Talley et al., including for example the compound 4-[5-(4-methylphenyl)-3-(trifluoromethyl)-1H-pyrazol-1-yl]benzenesulfonamide, also referred to herein as celecoxib (I), and the compound 4-[5-(3-fluoro-4-methoxyphenyl)-3-difluoromethyl)-1H-pyrazol-1-yl]benzenesulfonamide, also referred to herein as deracoxib (II).

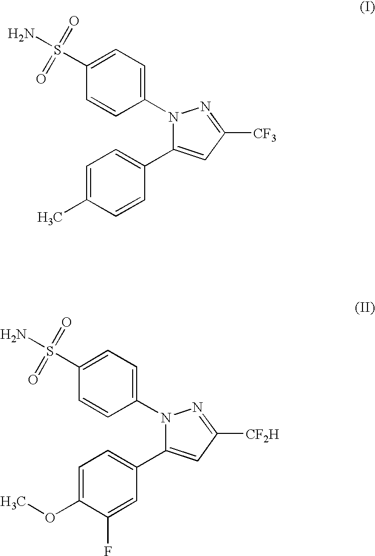

Other compounds reported to have therapeutically and/or prophylactically useful selective COX-2 inhibitory effect are substituted isoxazolyl benzenesulfonamides as reported in U.S. Pat. No. 5,633,272 to Talley et al., including for example the compound 4-[5-methyl-3-phenylisoxazol-4-yl]benzenesulfonamide, also referred to herein as valdecoxib (III).

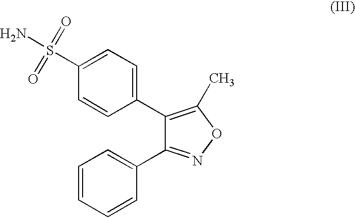

Still other compounds reported to have therapeutically and/or prophylactically useful selective COX-2 inhibitory effect are substituted (methylsulfonyl)phenyl furanones as reported in U.S. Pat. No. 5,474,995 to Ducharme et al., including for example the compound 3-phenyl-4-[4-(methylsulfonyl)phenyl]-5H-furan-2-one, also referred to herein as rofecoxib (IV).

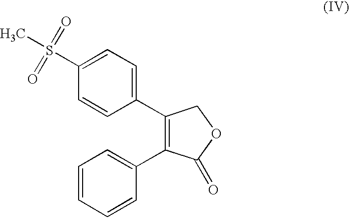

U.S. Pat. No. 5,981,576 to Belley et al. discloses a further series of (methylsulfonyl)phenyl furanones said to be useful as selective COX-2 inhibitory drugs, including 3-(1-cyclopropylmethoxy)-5,5-dimethyl-4-[4-(methylsulfonyl)phenyl]-5H-furan-2-one and 3-(1-cyclopropylethoxy)-5,5-dimethyl-4-[4-(methylsulfonyl)phenyl]-5H-furan-2-one.

U.S. Pat. No. 5,861,419 to Dube et al. discloses substituted pyridines said to be useful as selective COX-2 inhibitory drugs, including for example the compound 5-chloro-3-(4-methylsulfonyl)phenyl-2-(2-methyl-5-pyridinyl)pyridine, also referred to herein as etoricoxib (V).

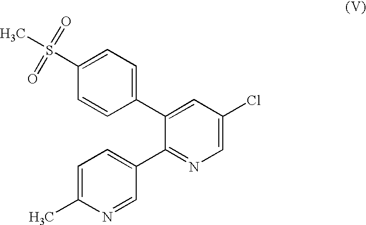

European Patent Application No. 0 863 134 discloses the compound 2-(3,5-difluorophenyl)-3-[4-(methylsulfonyl)phenyl]-2-cyclopenten-1-one said to be useful as a selective COX-2 inhibitory drug. International Patent Publication No. WO 99/11605 discloses 5-alkyl-2-arylaminophenylacetic acids and derivatives thereof, including the compound 5-methyl-2-(2′-chloro-6′-fluoroanilino)phenylacetic acid and salts thereof, said to be selective inhibitors of COX-2.

U.S. Pat. No. 6,034,256 to Carter et al. discloses a series of benzopyrans said to be useful as selective COX-2 inhibitory drugs, including the compound (S)-6,8-dichloro-2-(trifluoromethyl)-2H-1-benzopyran-3-carboxylic acid (VI).

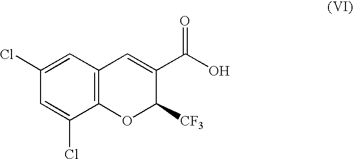

International Patent Publication No. WO 00/24719 discloses substituted pyridazinones said to be useful as selective COX-2 inhibitory drugs, including the compound 2-(3,4-difluorophenyl)-4-(3-hydroxy-3-methyl-1-butoxy)-5-[4-(methylsulfonyl)phenyl]-3-(2H)-pyridazinone.

Selective COX-2 inhibitory drugs have been formulated in a variety of ways, principally for oral delivery. However, topical administration of such drugs has been suggested in general terms, for example in some of the above-cited patents.

Above-cited U.S. Pat. No. 5,466,823 and No. 5,633,272 disclose that their subject compounds, which include celecoxib and valdecoxib, can be delivered topically. It is further disclosed in these patents that the compounds may be dissolved in water, polyethylene glycol, propylene glycol, ethanol, corn oil, cottonseed oil, peanut oil, sesame oil and benzyl alcohol.

Above-cited U.S. Pat. No. 5,474,995 discloses that its subject compounds, which include rofecoxib, can be formulated as creams, ointments, jellies, solutions or suspensions for topical use. Above-cited U.S. Pat. No. 5,861,419 similarly discloses that its subject compounds, which include etoricoxib, can be formulated as creams, ointments, jellies, solutions or suspensions for topical use, and further suggests that topical formulations may generally be comprised of a pharmaceutical carrier, co-solvent, emulsifier, penetration enhancer, preservative system and emollient.

Above-cited U.S. Pat. No. 6,034,256 discloses that its subject compounds, which include (S)-6,8-dichloro-2-(trifluoromethyl)-2H-1-benzopyran-3-carboxylic acid and salts thereof, can be applied as a topical ointment or cream for treatment of inflammations of external tissues, e.g., skin.

U.S. Pat. No. 5,932,598 to Talley et al. discloses a class of water-soluble prodrugs of selective COX-2 inhibitory drugs, including the compound N-[[4-(5-methyl-3-phenylisoxazol-4-yl)phenyl]sulfonyl]propanamide, also referred to herein as parecoxib (VII), and salts thereof, for example the sodium salt, referred to herein as parecoxib sodium. Parecoxib converts to the substantially water-insoluble selective COX-2 inhibitory drug valdecoxib following administration to a subject. Parecoxib itself shows weak in vitro inhibitory activity against both COX-1 and COX-2, while valdecoxib (II) has strong inhibitory activity against COX-2 but is a weak inhibitor of COX-1.

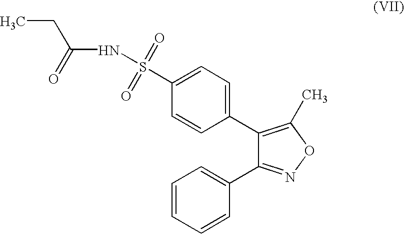

Because of the high water solubility of parecoxib, particularly of salts such as parecoxib sodium, by comparison with most selective COX-2 inhibitory drugs such as celecoxib and valdecoxib, the prodrug parecoxib has been proposed for parenteral use. See Talley et al. (2000), J. Med. Chem. 43, 1661-1663.

Above-cited U.S. Pat. No. 5,932,598 and No. 6,034,256 disclose that their subject compounds can be applied as a topical ointment or cream for treatment of inflammations of external tissues, e.g., skin. It is further disclosed therein that the aqueous phase of a cream base for such purpose may include at least 30% by weight of a polyhydric alcohol such as propylene glycol, butane-1,3-diol, mannitol, sorbitol, glycerol, polyethylene glycol and mixtures thereof, and that the topical formulation may include a dermal penetration enhancer such as dimethylsulfoxide. It is still further disclosed therein that the subject compounds can be administered by a transdermal device, for example using a patch either of the reservoir and porous membrane type or of a solid matrix variety.

U.S. Pat. No. 5,607,690 to Akizawa discloses an external anti-inflammatory and analgesic plaster preparation containing the NSAID diclofenac in the form of its hydroxyethylpyrrolidine salt, which is reported to exhibit enhanced skin permeation by comparison with an otherwise similar preparation containing diclofenac sodium. The low skin permeability of diclofenac sodium is stated therein to result from the low solubility in water of this salt.

International Patent Publication No. WO 99/62557 discloses a composition for transdermal administration of an NSAID comprising an absorption promoter that consists essentially of a diethylene glycol ether and a sorbitan ester, and an adhesive matrix.

International Patent Publication No. WO 00/41538 discloses a composition for transdermal administration of a drug comprising a blend of two or more acrylic-based polymers having differing functionalities.

International Patent Publication No. WO 00/51575 discloses a transdermal device containing a composition of an NSAID with a skin permeation enhancer selected from fatty alcohols, e.g., oleyl alcohol and fatty acid esters, e.g., glyceryl monooleate, isopropyl myristate.

International Patent Publication No. WO 97/29735 discloses a transdermal drug delivery system comprising a dermal penetration enhancer that is an ester sunscreen, preferably a long-chain alkyl ester of p-aminobenzoic acid, dimethyl p-aminobenzoic acid, cinnamic acid, methoxycinnamic acid or salicylic acid, for example octyl dimethyl p-aminobenzoate or octyl salicylate.

Administration of an NSAID, and more particularly of a selective COX-2 inhibitory drug, to the skin with the objective of achieving local or systemic therapeutic effect has therefore been widely contemplated in the art. However, there remains a need in the art for a selective COX-2 inhibitory drug composition that can be shown to exhibit a sufficient rate of skin permeation of the drug to achieve such effect.

Where a systemic effect is desired, the composition must be capable of delivering daily an amount of the drug by skin permeation at least equal to the minimum therapeutically effective daily dosage amount when the drug is given orally or parenterally. Furthermore, it is neither practical nor convenient to apply a drug over a very large area of skin to achieve this result; typically a maximum area for application is about 400 cm², but preferably a much smaller area of skin is treated.

For illustration, in the case of celecoxib, a typical minimum daily dosage amount by oral administration for an adult human is about 200 mg. A minimum permeation rate of 500 μg/cm².day over an area of 400 cm² is therefore needed to provide the minimum daily dosage amount of celecoxib. It is generally desirable to treat a much smaller area than 400 cm², thus the minimum permeation rate desired is even higher than 500 μg/cm².day. Even where only local delivery is desired, a high permeation rate is still important, because the area of skin available for local application is generally no greater than about 140 cm², often much less. In practice, a permeation rate of at least about 10 μg/cm².day, even for the most therapeutically potent selective COX-2 inhibitory drugs, is desirable in the great majority of situations.

Whether a systemic or local therapeutic effect is desired, it has therefore remained a difficult challenge to formulate a selective COX-2 inhibitory drug composition having therapeutic effectiveness when administered to an area of skin no greater than about 400 cm².

### SUMMARY OF THE INVENTION

There is now provided a dermally deliverable pharmaceutical composition comprising a therapeutic agent in a therapeutically effective amount solubilized in a solubilizing amount of a pharmaceutically acceptable carrier that comprises a low molecular weight monohydric alcohol, wherein (a) the therapeutic agent comprises at least one selective COX-2 inhibitory drug or prodrug thereof, and (b) a test sample of the composition provides a skin permeation rate of the therapeutic agent at least equal to that provided by a reference solution of the therapeutic agent in 70% aqueous ethanol.

A “reference solution” herein is one having the same concentration of the therapeutic agent as the test sample, up to the limit of solubility of the therapeutic agent in 70% aqueous ethanol. Such a reference solution is itself an embodiment of the present invention.

Preferably a skin permeation rate of not less than about 10 μg/cm².day is provided by the test sample.

There is further provided a dermally deliverable pharmaceutical composition comprising a therapeutic agent solubilized in a solubilizing amount of a pharmaceutically acceptable carrier that comprises a low molecular weight monohydric alcohol, wherein the therapeutic agent comprises at least one selective COX-2 inhibitory drug or prodrug thereof and is present at a concentration in the composition of about 12.5 to about 400 mg/ml.

There is still further provided a dermally deliverable pharmaceutical composition comprising a therapeutic agent solubilized in a solubilizing amount of a pharmaceutically acceptable carrier that comprises a low molecular weight monohydric alcohol, wherein the therapeutic agent comprises valdecoxib and/or a prodrug thereof and is present at a concentration in the composition of about 0.5 to about 400 mg/ml.

In preferred compositions of the invention, the carrier further comprises a skin permeation enhancer.

There is still further provided a method of effecting targeted delivery of a selective COX-2 inhibitory drug to a site of pain and/or inflammation in a subject, the method comprising topically administering a pharmaceutical composition as provided herein to a skin surface of the subject, preferably at a locus overlying or adjacent to the site of pain and/or inflammation.

There is still further provided a method of effecting systemic treatment of a subject having a COX-2 mediated disorder, the method comprising transdermally administering a pharmaceutical composition as provided herein, preferably by contacting the composition with an area of skin of the subject not greater than about 400 cm².

### DETAILED DESCRIPTION OF THE INVENTION

A dermally deliverable pharmaceutical composition of the invention comprises a therapeutic agent solubilized in a solubilizing amount of a pharmaceutically acceptable carrier that comprises a low molecular weight monohydric alcohol. For example, the therapeutic agent can be present at an unsaturated, saturated or supersaturated concentration, so long as the therapeutic agent remains in solubilized form for an acceptable time period between preparation and use when stored in a closed container at normal ambient temperature.

What constitutes an “acceptable time period” is situation dependent, but is normally at least about 5 days, preferably at least about 30 days, more preferably at least about 6 months, still more preferably at least about 1 year, and most preferably at least about 2 years.

Optionally, in addition to a solubilized component of the therapeutic agent as required herein, there can be a second component of the therapeutic agent that is present in particulate form, dispersed in the carrier, for example in stable suspension therein. This second component can act as a reservoir of the therapeutic agent to maintain substantial saturation of the solubilized component. However, it is generally preferred that substantially all of the therapeutic agent is present in solubilized form.

The term “dermally deliverable” means that the composition is suitable for direct application to skin and permits absorption into the skin and/or permeation through the skin of the agent in an amount sufficient to provide local and/or systemic therapeutic effect.

The therapeutic agent comprises at least one selective COX-2 inhibitory drug or prodrug thereof. Any such selective COX-2 inhibitory drug or prodrug known in the art can be used.

A preferred selective COX-2 inhibitory drug useful herein is a compound of formula (VIII):

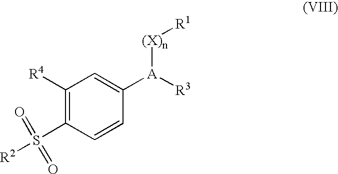

or a prodrug or pharmaceutically acceptable salt thereof, wherein:

A is a substituent selected from partially unsaturated or unsaturated heterocyclyl and partially unsaturated or unsaturated carbocyclic rings, preferably a heterocyclyl group selected from pyrazolyl, furanonyl, isoxazolyl, pyridinyl, cyclopentenonyl and pyridazinonyl groups;

X is O, S or CH₂;

n is 0 or 1;

R¹ is at least one substituent selected from heterocyclyl, cycloalkyl, cycloalkenyl and aryl, and is optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio;

R² is methyl, amino or aminocarbonylalkyl;

R³ is one or more radicals selected from hydrido, halo, alkyl, alkenyl, alkynyl, oxo, cyano, carboxyl, cyanoalkyl, heterocyclyloxy, alkyloxy, alkylthio, alkylcarbonyl, cycloalkyl, aryl, haloalkyl, heterocyclyl, cycloalkenyl, aralkyl, heterocyclylalkyl, acyl, alkylthioalkyl, hydroxyalkyl, alkoxycarbonyl, arylcarbonyl, aralkylcarbonyl, aralkenyl, alkoxyalkyl, arylthioalkyl, aryloxyalkyl, aralkylthioalkyl, aralkoxyalkyl, alkoxyaralkoxyalkyl, alkoxycarbonylalkyl, aminocarbonyl, aminocarbonylalkyl, alkylaminocarbonyl, N-arylaminocarbonyl, N-alkyl-N-arylaminocarbonyl, alkylaminocarbonylalkyl, carboxyalkyl, alkylamino, N-arylamino, N-aralkylamino, N-alkyl-N-aralkylamino, N-alkyl-N-arylamino, aminoalkyl, alkylaminoalkyl, N-arylaminoalkyl, N-aralkylaminoalkyl, N-alkyl-N-aralkylaminoalkyl, N-alkyl-N-arylaminoalkyl, aryloxy, aralkoxy, arylthio, aralkylthio, alkylsulfinyl, alkylsulfonyl, aminosulfonyl, alkylaminosulfonyl, N-arylaminosulfonyl, arylsulfonyl and N-alkyl-N-arylaminosulfonyl, R³ being optionally substituted at a substitutable position with one or more radicals selected from alkyl, haloalkyl, cyano, carboxyl, alkoxycarbonyl, hydroxyl, hydroxyalkyl, haloalkoxy, amino, alkylamino, arylamino, nitro, alkoxyalkyl, alkylsulfinyl, halo, alkoxy and alkylthio; and

R⁴ is selected from hydrido and halo.

Compositions of the invention are especially useful for selective COX-2 inhibitory drugs having the formula (IX):

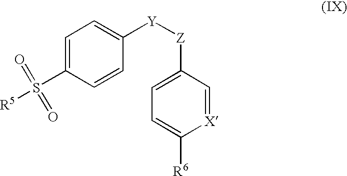

where R⁵ is a methyl or amino group, R⁶ is hydrogen or a C₁₋₄ alkyl or alkoxy group, X′ s N or CR⁷ where R⁷ is hydrogen or halogen, and Y and Z are independently carbon or nitrogen atoms defining adjacent atoms of a five- to six-membered ring that is optionally substituted at one or more positions with oxo, halo, methyl or halomethyl groups, or an isomer, tautomer, pharmaceutically-acceptable salt or prodrug thereof. Preferred such five- to six-membered rings are cyclopentenone, furanone, methylpyrazole, isoxazole and pyridine rings substituted at no more than one position.

Compositions of the invention are also useful for compounds having the formula (X):

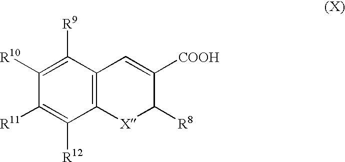

where X″ is O, S or N-lower alkyl; R⁸ is lower haloalkyl; R⁹ is hydrogen or halogen; R¹⁰ is hydrogen, halogen, lower alkyl, lower alkoxy or haloalkoxy, lower aralkylcarbonyl, lower dialkylaminosulfonyl, lower alkylaminosulfonyl, lower aralkylaminosulfonyl, lower heteroaralkylaminosulfonyl, or 5- or 6- membered nitrogen-containing heterocyclosulfonyl; and R¹¹ and R¹² are independently hydrogen, halogen, lower alkyl, lower alkoxy, or aryl; and for pharmaceutically acceptable salts thereof.

A particularly useful compound of formula (X) is (S)-6,8-dichloro-2-(trifluoromethyl)-2H-1-benzopyran-3-carboxylic acid.

Compositions of the invention are also useful for selective COX-2 inhibitory 5-alkyl-2-arylaminophenylacetic acids and derivatives thereof. Particularly useful compounds of this class are 5-methyl-2-(2′-chloro-6′-fluoroanilino)phenylacetic acid and pharmaceutically acceptable salts thereof.

Illustratively, celecoxib, deracoxib, valdecoxib, parecoxib, rofecoxib, etoricoxib, 2-(3,5-difluorophenyl)-3-[4-(methylsulfonyl)phenyl]-2-cyclopenten-1-one, (S)-6,8-dichloro-2-(trifluoromethyl)-2H-1-benzopyran-3-carboxylic acid, 2-(3,4-difluorophenyl)-4-(3-hydroxy-3-methyl-1-butoxy)-5-[4-(methylsulfonyl)phenyl]-3-(2H)-pyridazinone, 5-methyl-2-(2′-chloro-6′-fluoroanilino)phenylacetic acid and their salts, more particularly celecoxib, valdecoxib, parecoxib and its salts, rofecoxib and etoricoxib, are useful in the method and composition of the invention.

In a presently preferred embodiment, the therapeutic agent comprises valdecoxib and/or a prodrug thereof, for example parecoxib and/or a salt thereof, such as parecoxib sodium.

Valdecoxib used in compositions of the invention can be prepared by any known process, for example in the manner set forth in above-cited U.S. Pat. No. 5,633,272. Parecoxib used in compositions of the invention can be prepared by any known process, for example in the manner set forth in above-cited U.S. Pat. No. 5,932,598. Other selective COX-2 inhibitory drugs can be prepared by any known process, including processes set forth in patent publications disclosing such drugs; for example in the case of celecoxib in above-cited U.S. Pat. No. 5,466,823 or in U.S. Pat. No. 5,892,053 to Zhi et al., incorporated herein by reference.

According to a first embodiment of the invention, the composition exhibits a skin permeation rate of the therapeutic agent at least equal to that provided by a reference solution of the therapeutic agent in 70% aqueous ethanol, preferably a rate of not less than about 10 μg/cm².day. When a skin permeation rate or range of such rates is indicated herein, it will be understood to mean a rate as determined by a standard test, illustratively a standard test using human cadaver skin.

As an example of such a test, a Franz diffusion cell can be used having a cadaver skin membrane of suitable area, e.g., a disk of diameter 20 mm, and a suitable receptor fluid, as described more particularly in the Examples below. Suitable receptor fluids can be selected by one of skill in the art, but presently preferred receptor fluids are a 1% polysorbate 80 solution and a 6% polyethylene glycol (20) oleyl ether (oleth-20) solution. The receptor fluid is maintained at a suitable temperature, preferably a temperature approximating living human skin temperature. A receptor fluid temperature of 32° C. has been found suitable. The membrane is oriented so that its internal surface, i.e., the surface opposite the epidermal surface, is placed in contact with the receptor fluid. Air bubbles are removed from the receptor fluid, which is then allowed to equilibrate for 30 minutes with the membrane. A test sample of a composition is placed in contact with the epidermal surface of the membrane, and left in place for a desired period, for example 24 hours. At intervals during this period, and/or at the end of this period, concentration of one or more selective COX-2 inhibitory drugs is determined in the receptor fluid by a suitable analytical method, e.g., high performance liquid chromatography (HPLC). This concentration is a measure of the amount of the drug or drugs that have permeated the skin membrane during the period of the test, and can be used to calculate a skin permeation rate of drug in units such as μg/cm².day or μg/cm².hour.

It will be understood that skin membranes exhibit significant variation in permeability, depending on source. Absolute permeation rates through such membranes are therefore less meaningful than relative permeation rates by comparison with a reference composition. A standard reference composition adopted herein is a solution of the therapeutic agent in 70% aqueous ethanol. It has been found that such a reference composition, which is itself an embodiment of the invention, commonly provides a skin permeation rate of about 10 μg/cm².day or greater, particularly if the therapeutic agent is in substantially saturated solution in the 70% aqueous ethanol. However, a composition is not excluded from the scope of the present invention if in a test using a particular skin membrane it provides a skin permeation rate lower than 10 μg/cm².day, so long as the rate is at least equal to that exhibited in a comparative test of the reference composition using a skin membrane from the same source.

Preferred permeation rates depend to some extent on the therapeutic potency of the drug or prodrug selected. In the case of celecoxib, for example, which requires relatively high blood levels for therapeutic effectiveness, the skin permeation rate is preferably not less than about 25 μg/cm².day, more preferably not less than about 50 μg/cm².day, still more preferably not less than about 75 μg/cm².day and most preferably not less than about 100 μg/cm².day.

According to a second embodiment of the invention, the therapeutic agent in the composition comprises at least one selective COX-2 inhibitory drug or prodrug thereof and is present in the composition at a concentration of about 12.5 to about 400 mg/ml. Below this concentration range, for example at a concentration of 10 mg/ml (or about 1% by weight) the skin permeation rate for most selective COX-2 inhibitory drugs, even in the presence of a permeation enhancer, is likely to be too low to be therapeutically effective. Above this concentration range, for example at a concentration of about 40% by weight (which depending on the specific gravity of the composition can be equivalent to a concentration of about 420 to about 500 mg/ml), it is likely to be very difficult to solubilize most selective COX-2 inhibitory drugs, prodrugs or salts thereof.

Preferably in this embodiment the concentration of the therapeutic agent is about 12.5 to about 375 mg/ml, more preferably 12.5 to about 250 mg/ml and most preferably about 12.5 to about 125 mg/ml. It will be understood by one of skill in the art that for a drug having a relatively high dosage requirement (e.g., celecoxib) the optimum concentration is likely to be higher than for a drug having a relatively low dosage requirement (e.g., valdecoxib).

Celecoxib compositions of the present invention, to be useful for transdermal application to give systemic delivery of the drug, preferably contain celecoxib in a concentration permitting a daily dosage amount of about 100 mg to about 400 mg, for example about 250 mg to about 350 mg, illustratively about 275 mg to about 325 mg. Preferably the concentration is such that this dosage amount can be provided by application of the composition one to four times a day, to a skin area of up to about 400 cm².

Valdecoxib compositions of the present invention, to be useful for transdermal application to give systemic delivery of the drug, preferably contain valdecoxib in a concentration permitting a daily dosage amount of about 10 mg to about 100 mg, preferably about 20 mg to about 80 mg, for example about 30 mg to about 40 mg, illustratively about 32 mg to about 38 mg, more particularly about 34 mg to about 36 mg. Preferably the concentration is such that this dosage amount can be provided by application of the composition one to four times a day, preferably one to two times a day, to a skin area of up to about 400 cm², preferably about 1 cm² to about 100 cm².

Parecoxib compositions of the present invention, to be useful for transdermal application to give systemic delivery of valdecoxib, preferably contain parecoxib or a salt thereof in a concentration permitting a daily dosage amount of about 10 mg to about 100 mg, preferably about 30 mg to about 80 mg, for example about 45 mg to about 75 mg, illustratively about 50 mg to about 70 mg. Preferably the concentration is such that this dosage amount can be provided by application of the composition one to four times a day, preferably one to two times a day, to a skin area of up to about 400 cm², preferably about 1 cm² to about 100 cm².

For other selective COX-2 inhibitory drugs and prodrugs, the concentration should provide a daily dosage amount in a range known to be therapeutically effective for such drugs and prodrugs. Preferably, the daily dosage amount is in a range providing therapeutic equivalence to celecoxib, valdecoxib or parecoxib in the daily dose ranges indicated immediately above.

According to a third embodiment of the invention, the therapeutic agent in the composition comprises valdecoxib and/or a prodrug thereof and is present in the composition at a concentration of about 0.5 to about 400 mg/ml, preferably about 0.5 to about 125 mg/ml. Concentration of the therapeutic agent by weight in this embodiment is preferably about 0.05% to about 10%, more preferably about 0.5% to about 5%, particularly where the composition is to be used to effect targeted delivery of the therapeutic agent to a site of pain and/or inflammation from an overlying or adjacent skin surface.

In this third embodiment, a preferred prodrug is parecoxib or a salt thereof, for example parecoxib sodium.

Alternatively according to this third embodiment, the therapeutic agent can be valdecoxib alone or in combination with another drug.

It has surprisingly been found that a composition comprising both parecoxib or a salt thereof and a selective COX-2 inhibitory drug of low water solubility, for example celecoxib or valdecoxib, the skin permeation rate of the drug of low water solubility is greatly increased by comparison with a composition lacking the parecoxib. Thus, in a particular embodiment, the therapeutic agent in a composition as described above comprises parecoxib or a salt thereof and a selective COX-2 inhibitory drug of low water solubility. According to this embodiment, the drug of low water solubility can illustratively be selected from celecoxib, deracoxib, valdecoxib, rofecoxib, etoricoxib, 2-(3,5-difluorophenyl)-3-[4-(methylsulfonyl)phenyl]-2-cyclopenten-1-one and 2-(3,4-difluorophenyl)-4-(3-hydroxy-3-methyl-1-butyoxy)-5-[4-(methylsulfonyl)phenyl]-3-(2H)-pyridazinone.

According to any of the above embodiments, the therapeutic agent is preferably fully solubilized in the carrier.

The carrier comprises a pharmaceutically acceptable solvent for the therapeutic agent. For a therapeutic agent consisting of one or more water-soluble drugs or prodrugs, for example parecoxib sodium, water is a preferred solvent. For drugs or prodrugs of low water solubility, one or more pharmaceutically acceptable organic solvents will be required. Such solvents can, for example, be selected from mono-, di- and polyhydric alcohols, illustratively including ethanol, isopropanol, n-butanol, 1,3-butanediol, propylene glycol, glycerol, glycofurol, myristyl alcohol, oleyl alcohol and polyethylene glycol (PEG), e.g., PEG having an average molecular weight of about 200 to about 800. Suitable PEGs include PEG-200, PEG-350, PEG-400, PEG-540 and PEG-600, with PEG-400 being preferred. Some of the above solvents can function additionally as skin permeation enhancers.

Alternatively or in addition, a pharmaceutically acceptable glycol ether solvent can be used, such as those conforming to formula (XI):

R¹—O—((CH₂)ₘO)ₙ—R² (XI)

wherein R¹ and R² are independently hydrogen or C₁₋₆ alkyl, C₁₋₆ alkenyl, phenyl or benzyl groups, but no more than one of R₁ and R₂ is hydrogen; m is an integer of 2 to about 5; and n is an integer of 1 to about 20. It is preferred that one of R¹ and R² is a C₁₋₄ alkyl group and the other is hydrogen or a C₁₋₄ alkyl group; more preferably at least one of R¹ and R² is a methyl or ethyl group. It is preferred that m is 2. It is preferred that n is an integer of 1 to about 4, more preferably 2.

Glycol ethers useful in compositions of the present invention typically have a molecular weight of about 75 to about 1000, preferably about 75 to about 500, and more preferably about 100 to about 300. Importantly, such glycol ethers must be pharmaceutically acceptable and must meet all other conditions prescribed herein.

Non-limiting examples of glycols and glycol ethers that may be used in compositions of the present invention include ethylene glycol monomethyl ether, ethylene glycol dimethyl ether, ethylene glycol monoethyl ether, ethylene glycol diethyl ether, ethylene glycol monobutyl ether, ethylene glycol dibutyl ether, ethylene glycol monophenyl ether, ethylene glycol monobenzyl ether, ethylene glycol butylphenyl ether, ethylene glycol terpinyl ether, diethylene glycol monomethyl ether, diethylene glycol dimethyl ether, diethylene glycol monoethyl ether, diethylene glycol diethyl ether, diethylene glycol divinyl ether, ethylene glycol monobutyl ether, diethylene glycol dibutyl ether, diethylene glycol monoisobutyl ether, triethylene glycol dimethyl ether, triethylene glycol monoethyl ether, triethylene glycol monobutyl ether, tetraethylene glycol dimethyl ether, and mixtures thereof. See for example Flick (1998): Industrial Solvents Handbook, 5th ed., Noyes Data Corporation, Westwood, N.J.

A presently preferred glycol ether solvent is diethylene glycol monoethyl ether, sometimes referred to in the art as DGME or ethoxydiglycol. It is available for example under the trademark Transcutol™ of Gattefossé Corporation.

According to the present invention, at least one solvent or skin permeation enhancer present is a low molecular weight monohydric alcohol. By “low molecular weight” in this context is meant having substantially lower molecular weight than myristyl alcohol. Preferred low molecular weight monohydric alcohols are C₂₋₆ monohydric alcohols, for example ethanol, isopropanol, n-butanol or DGME.

It has surprisingly been found that an ethanol-water mixture as solvent for a selective COX-2 inhibitory drug such as celecoxib or valdecoxib generally gives a greater skin permeation rate of the drug than ethanol alone. Suitable weight ratios of ethanol to water are from about 50/50 to about 90/10. An optimum ratio is about 65/35 to about 75/25, for example about 70/30. Thus a composition having a carrier consisting of ethanol alone will typically not meet the criterion established herein of providing a skin permeation rate at least equal to that provided by a reference solution of the therapeutic agent in 70% aqueous ethanol.

Compositions of the present invention optionally comprise one or more pharmaceutically acceptable co-solvents. Non-limiting examples of co-solvents suitable for use in compositions of the present invention include any solvent listed above; N-methyl-2-pyrrolidinone (NMP); oleic and linoleic acid triglycerides, for example soybean oil; caprylic/capric triglycerides, for example Miglyol™ 812 of Huls; caprylic/capric mono- and diglycerides, for example Capmul™ MCM of Abitec; benzyl phenylformate; diethyl phthalate; triacetin; polyoxyethylene caprylic/capric glycerides such as polyoxyethylene (8) caprylic/capric mono- and diglycerides, for example Labrasol™ of Gattefossé; medium chain triglycerides; propylene glycol fatty acid esters, for example propylene glycol laurate; oils, for example corn oil, mineral oil, cottonseed oil, peanut oil, sesame seed oil and polyoxyethylene (35) castor oil, for example Cremophor™ EL of BASF; polyoxyethylene glyceryl trioleate, for example Tagat™ TO of Goldschmidt; polyoxyethylene sorbitan esters, for example polysorbate 80; and lower alkyl esters of fatty acids, for example ethyl butyrate, ethyl caprylate and ethyl oleate.

It is preferred to include as a component of the carrier a skin permeation enhancer.

In one embodiment, a permeation enhancer selected from terpenes, terpenoids, fatty alcohols and derivatives thereof is present in the carrier. Examples include oleyl alcohol, thymol, menthol, carvone, carveol, citral, dihydrocarveol, dihydrocarvone, neomenthol, isopulegol, 4-terpinenol, menthone, pulegol, camphor, geraniol, α-terpineol, linalool, carvacrol, trans-anethole, isomers thereof and racemic mixtures thereof. Optionally more than one such permeation enhancer, for example a fatty alcohol and a terpene or terpenoid, can be present. Thus, in an illustrative embodiment, a composition of the invention comprises as penetration enhancers oleyl alcohol and thymol.

Fatty acids such as oleic acid and their alkyl and glyceryl esters such as isopropyl laurate, isopropyl myristate, methyl oleate, glyceryl monolaurate, glyceryl monooleate, glyceryl dilaurate, glyceryl dioleate, etc. also can be used as permeation enhancers. Fatty acid esters of glycolic acid and its salts, for example as disclosed in International Patent Publication No. WO 98/18416, incorporated herein by reference, are also useful permeation enhancers. Examples of such esters include lauroyl glycolate, caproyl glycolate, cocoyl glycolate, isostearoyl glycolate, sodium lauroyl glycolate, tromethamine lauroyl glycolate, etc. Also useful as permeation enhancers are lactate esters of fatty alcohols, for example lauryl lactate, myristyl lactate, oleyl lactate, etc. An example of a particularly preferred permeation enhancer is glyceryl monolaurate.

Other permeation enhancers include hexahydro-1-dodecyl-2H-azepin-2-one (laurocapram, Azone™) and derivatives thereof, dimethylsulfoxide (DMSO), n-decyl methylsulfoxide, salicylic acid and alkyl esters thereof, e.g., methyl salicylate, N,N-dimethylacetamide, dimethylformamide, N,N-dimethyltoluamide, 2-pyrrolidinone and N-alkyl derivatives thereof, e.g., NMP and N-octyl-2-pyrrolidinone, 2-nonyl-1,3-dioxolane, eucalyptol and sorbitan esters.

An illustrative carrier comprises DMSO and water in a ratio of 100:0 to about 10:90 by volume.

Another illustrative carrier comprises oleyl alcohol and propylene glycol in a ratio of about 20:80 to about 5:95 by volume.

Yet another illustrative carrier comprises laurocapram and propylene glycol in a ratio of about 20:80 to about 5:95 by volume.

In a particular embodiment, the carrier comprises as a permeation enhancer a sunscreen. This can be an ester sunscreen as described, for example, in above-cited International Patent Publication No. WO 97/29735, incorporated herein by reference. Preferred permeation enhancers according to this embodiment are compounds of formula (XII):

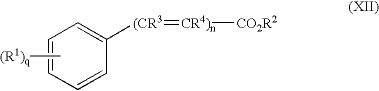

where R¹ groups are independently hydrogen, lower alkyl, lower alkoxy, halogen, hydroxyl or NR⁵R⁶ groups in which R⁵ and R⁶ are independently hydrogen or lower alkyl groups or R⁵ and R⁶ together with the nitrogen atom to which they are attached form a 5- or 6-membered heterocyclic ring; R² is a C₅₋₁₈ linear, branched or cyclic alkyl group; R³ is a hydrogen or phenyl group; R⁴ is a hydrogen or cyano group; n is 0 or 1; and q is 1 or 2. R² in a compound of formula (XII) is preferably a C₅₋₁₂ alkyl group, most preferably an isoamyl, octyl (i.e., 2-ethylhexyl), menthyl or homomenthyl group.

Particularly preferred compounds of formula (XII) are alkyl esters of p-aminobenzoic acid (PABA), p-dimethylaminobenzoic acid, 2-aminobenzoic acid, cinnamic acid, p-methoxycinnamic acid, salicylic acid and 2-cyano-3,3-diphenylacrylic acid, for example 2-ethylhexyl p-dimethylaminobenzoate (Padimate O), 2-ethylhexyl p-methoxycinnamate, 2-ethylhexyl salicylate, menthyl salicylate, homomenthyl salicylate (homosalate), menthyl 2-aminobenzoate and 2-ethylhexyl 2-cyano-3,3-diphenylacrylate (octocrylene).

Compounds of formula (XII) are useful as permeation enhancers herein even if they are not effective as sunscreens.

Alternatively the sunscreen can be other than an ester sunscreen, for example a benzophenone sunscreen or modification thereof, such as 2-hydroxy-4-methoxybenzophenone (oxybenzone), 2,2′-dihydroxy-4-methoxybenzophenone (dioxybenzone), 5-benzoyl-4-hydroxy-2-methoxybenzenesulfonic acid (sulisobenzone) or 1-(p-tert-butylphenyl)-3-(p-methoxyphenyl)-1,3-propanedione (avobenzone).

Other ingredients of the carrier can include one or more excipients selected from thickening agents, surfactants, emulsifiers, antioxidants, preservatives, stabilizers, colors and fragrances. A skin irritation reducing agent, such as vitamin E, glycyrrhetic acid or diphenhydramine, can also be present.

A composition of the invention can be in any liquid or semi-solid dosage form suitable for topical application to skin and can be formulated according to conventional methods known in the art. A dosage form as contemplated herein is one that does not have as a component thereof a solid backing material, although, following application of the composition to skin, an occluding material such as a dressing or bandage can, if desired, be applied over the treated area without removing the composition or method of treatment thereof from the scope of the present invention. A liquid or semi-solid dosage form of the invention can comprise a solution, a suspension and/or an emulsion.

A suitable dosage form can be for example a cream, paste, gel, ointment, lotion or aerosol. The concentration of therapeutic agent in the dosage form depends on the selective COX-2 inhibitory drug(s) or prodrug(s) in question, the desired dosage amount of such drug(s) or prodrug(s) to be administered, the desired frequency of administration, the selection of permeation enhancer if any, the nature of the dosage form and other factors.

A non-limiting illustrative paste, ointment, gel or cream is a composition of the invention comprising at least one selective COX-2 inhibitory drug or prodrug, at least one solvent, at least one skin permeation enhancer and at least one thickening agent. Suitable thickening agents for ointments, gels and creams include without limitation hydroxypropylcellulose, hydroxypropylmethylcellulose (HPMC), hydroxyethylcellulose, ethylcellulose, carboxymethylcellulose, dextran, guar gum, polyvinylpyrrolidone (PVP), pectin, starch, gelatin, casein, acrylic acid, acrylic acid esters, acrylic acid copolymers, vinyl alcohols, alkoxy polymers, polyethylene oxide polymers, polyethers and the like.

Illustratively such a composition can comprise amounts of these ingredients as follows (all percentages by weight):

| | |
|---|---|
| selective COX-2 inhibitory drug or prodrug | 1.25-10% |
| solvent(s) (e.g., 70% ethanol, 30% water) | 50-97% |
| co-solvent(s) and/or surfactant(s) | 0-15% |
| skin permeation enhancer(s) | 2-20% |
| thickening agent(s) | 1-5% |

Where the skin permeation enhancers comprise a fatty alcohol and a terpene or terpenoid, e.g., oleyl alcohol and thymol, suitable amounts of these in the illustrative composition described immediately above are 2-10% by weight of the fatty alcohol and 1-6% by weight of the terpene or terpenoid. Amounts outside these ranges can also be useful in particular situations.

Certain compounds listed above as permeation enhancers can function as topical analgesics in their own right. For example, methyl salicylate, menthol or a combination thereof (as found, for example, in Bengay® products of Pfizer) can provide complementary analgesia when included in a composition of the present invention. In particular, such compounds can provide early-onset, short-term analgesia that complements the longer-term, sustained analgesic and anti-inflammatory effects of the selective COX-2 inhibitory drug or prodrug. In compositions of the invention comprising methyl salicylate and menthol, suitable amounts are 5-30% by weight of methyl salicylate and 2-20% by weight of menthol. Amounts outside these ranges can also be useful in particular situations.

An embodiment of the invention is a composition suitable for application to skin by means of an applicator such as an aerosol, a spray, a pump-pack, a brush or a swab. Preferably, such an applicator provides fixed or variable metered dose application, as exemplified by a metered dose aerosol, a stored-energy metered dose pump or a manual metered dose pump. According to this embodiment, application is most preferably performed by means of a topical metered dose aerosol combined with an actuator nozzle shroud which together accurately control the amount and/or uniformity of the dose applied. The shroud can help control the distance of the nozzle from the skin, a function that can alternatively be achieved by means of a spacer-bar or the like. Another function of the shroud is to enclose the treated area of the skin in order to prevent or limit bounce-back and/or loss of the composition. Preferably the area of application defined by the shroud is substantially circular in shape. The composition may be propelled by a pump-pack or more preferably by use of an aerosol propellant such as a hydrocarbon or hydrofluorocarbon propellant, nitrogen, nitrous oxide, carbon dioxide or an ether, for example dimethyl ether.

In a particular embodiment, a cream, paste, gel, ointment, lotion or aerosol composition of the invention comprises as a skin permeation enhancer a sunscreen, e.g., octyl p-dimethylaminobenzoate (octyl dimethyl PABA or Padimate O). A suitable amount of such a sunscreen in the composition is 1-10%, preferably 2-8%, by weight.

In this embodiment the sunscreen can have a dual function as a sunscreen (i.e., protectant against sunburn or other ultraviolet injury to skin) and permeation enhancer for the selective COX-2 inhibitory drug or prodrug. Where the drug or prodrug is to be administered for relief of pain and/or inflammation arising from such injury, a composition of this embodiment can be especially useful. Optionally other typical ingredients of sunscreen preparations can be included, such as titanium dioxide.

A particular feature of the present invention is that the dosage form can be designed so that the drug penetrates the skin to deliver a therapeutically effective amount of the drug to a target site such as epidermal, dermal, subcutaneous, muscular and articular organs and tissues while maintaining systemic levels of the drug not greatly in excess of a minimum therapeutically effective level. Thus pharmaceutical compositions as described above can be used to effect targeted delivery of a selective COX-2 inhibitory drug to an external or internal site of pain and/or inflammation in a subject. According to a therapeutic method of the invention, a composition as provided herein is topically administered to a skin surface of the subject, preferably at a locus overlying or adjacent to the site of pain and/or inflammation.

Pharmaceutical compositions as described above can also be used to effect systemic treatment of a subject having a COX-2 mediated disorder. According to a therapeutic method of the invention, a pharmaceutical composition as provided herein is administered transdermally, preferably by contacting the composition with an area of skin of the subject not greater than about 400 cm².

In either of the above methods, the composition according to a first embodiment is a dermally deliverable pharmaceutical composition comprising a therapeutic agent solubilized in a pharmaceutically acceptable carrier that comprises a C₂₋₆ monohydric alcohol, wherein the therapeutic agent comprises at least one selective COX-2 inhibitory drug or prodrug thereof, and wherein the composition exhibits a skin permeation rate of the therapeutic agent at least equal to that exhibited by a reference solution of the therapeutic agent in 70% aqueous ethanol, preferably a rate of not less than about 10 μg/cm².day, more preferably not less than about 50 μg/cm².day.

In either of the above methods, the composition according to a second embodiment is a dermally deliverable pharmaceutical composition comprising a therapeutic agent solubilized in a pharmaceutically acceptable carrier that comprises a C₂₋₆ monohydric alcohol, wherein the therapeutic agent comprises at least one selective COX-2 inhibitory drug or prodrug thereof and is present at a concentration in the carrier of about 12.5 to about 400 mg/ml.

In either of the above methods, the composition according to a third embodiment is a dermally deliverable pharmaceutical composition comprising a therapeutic agent solubilized in a pharmaceutically acceptable carrier that comprises a C₂₋₆ monohydric alcohol, wherein the therapeutic agent comprises valdecoxib and/or a prodrug thereof and is present at a concentration in the carrier of about 0.5 to about 400 mg/ml.

Therapeutic methods and compositions of the invention are useful in treatment and prevention of a very wide range of disorders mediated by COX-2, including but not restricted to disorders characterized by inflammation, pain and/or fever. Such compositions are especially useful as anti-inflammatory agents, such as in treatment of arthritis, with the additional benefit of having significantly less harmful side effects than compositions of conventional non-steroidal anti-inflammatory drugs (NSAIDs) that lack selectivity for COX-2 over COX-1. In particular, compositions of the invention have reduced potential for gastrointestinal toxicity and gastrointestinal irritation including upper gastrointestinal ulceration and bleeding, reduced potential for renal side effects such as reduction in renal function leading to fluid retention and exacerbation of hypertension, reduced effect on bleeding times including inhibition of platelet function, and possibly a lessened ability to induce asthma attacks in aspirin-sensitive asthmatic subjects, by comparison with compositions of conventional NSAIDs. Thus compositions of the invention are particularly useful as an alternative to conventional NSAIDs where such NSAIDs are contraindicated, for example in patients with peptic ulcers, gastritis, regional enteritis, ulcerative colitis, diverticulitis or with a recurrent history of gastrointestinal lesions; gastrointestinal bleeding, coagulation disorders including anemia such as hypoprothrombinemia, hemophilia or other bleeding problems; kidney disease; or in patients prior to surgery or patients taking anticoagulants.

Contemplated compositions are useful to treat a variety of arthritic disorders, including but not limited to rheumatoid arthritis, spondyloarthropathies, gouty arthritis, osteoarthritis, systemic lupus erythematosus and juvenile arthritis.

Such compositions are useful in treatment of asthma, bronchitis, menstrual cramps, preterm labor, tendinitis, bursitis, allergic neuritis, cytomegalovirus infectivity, apoptosis including HIV-induced apoptosis, lumbago, liver disease including hepatitis, skin-related conditions such as psoriasis, eczema, acne, burns, dermatitis and ultraviolet radiation damage including sunburn, and post-operative inflammation including the following ophthalmic surgery such as cataract surgery or refractive surgery.

Such compositions are useful to treat gastrointestinal conditions such as inflammatory bowel disease, Crohn's disease, gastritis, irritable bowel syndrome and ulcerative colitis.

Such compositions are useful in treating inflammation in such diseases as migraine headaches, periarteritis nodosa, thyroiditis, aplastic anemia, Hodgkin's disease, sclerodoma, rheumatic fever, type I diabetes, neuromuscular junction disease including myasthenia gravis, white matter disease including multiple sclerosis, sarcoidosis, nephrotic syndrome, Behcet's syndrome, polymyositis, gingivitis, nephritis, hypersensitivity, swelling occurring after injury including brain edema, myocardial ischemia, and the like.

Such compositions are useful in treatment of ophthalmic diseases, such as retinitis, conjunctivitis, retinopathies, uveitis, ocular photophobia, and of acute injury to the eye tissue.

Such compositions are useful in treatment of pulmonary inflammation, such as that associated with viral infections and cystic fibrosis, and in bone resorption such as that associated with osteoporosis.

Such compositions are useful for treatment of certain central nervous system disorders, such as cortical dementias including Alzheimer's disease, neurodegeneration, and central nervous system damage resulting from stroke, ischemia and trauma. The term “treatment” in the present context includes partial or total inhibition of dementias, including Alzheimer's disease, vascular dementia, multi-infarct dementia, pre-senile dementia, alcoholic dementia and senile dementia.

Such compositions are useful in treatment of allergic rhinitis, respiratory distress syndrome, endotoxin shock syndrome and liver disease.

Such compositions are used in treatment of pain, including but not limited to postoperative pain, dental pain, muscular pain, and pain resulting from cancer. For example, such compositions are useful for relief of pain, fever and inflammation in a variety of conditions including rheumatic fever, influenza and other viral infections including common cold, low back and neck pain, dysmenorrhea, headache, toothache, sprains and strains, myositis, neuralgia, synovitis, arthritis, including rheumatoid arthritis, degenerative joint diseases (osteoarthritis), gout and ankylosing spondylitis, bursitis, bums, and trauma following surgical and dental procedures.

Such compositions are useful for treating and preventing inflammation-related cardiovascular disorders, including vascular diseases, coronary artery disease, aneurysm, vascular rejection, arteriosclerosis, atherosclerosis including cardiac transplant atherosclerosis, myocardial infarction, embolism, stroke, thrombosis including venous thrombosis, angina including unstable angina, coronary plaque inflammation, bacterial-induced inflammation including Chlamydia-induced inflammation, viral induced inflammation, and inflammation associated with surgical procedures such as vascular grafting including coronary artery bypass surgery, revascularization procedures including angioplasty, stent placement, endarterectomy, or other invasive procedures involving arteries, veins and capillaries.

Such compositions are useful in treatment of angiogenesis-related disorders in a subject, for example to inhibit tumor angiogenesis. Such compositions are useful in treatment of neoplasia, including metastasis; ophthalmological conditions such as corneal graft rejection, ocular neovascularization, retinal neovascularization including neovascularization following injury or infection, diabetic retinopathy, macular degeneration, retrolental fibroplasia and neovascular glaucoma; ulcerative diseases such as gastric ulcer; pathological, but non-malignant, conditions such as hemangiomas, including infantile hemangiomas, angiofibroma of the nasopharynx and avascular necrosis of bone; and disorders of the female reproductive system such as endometriosis.

Such compositions are useful in the treatment of pre-cancerous diseases, such as actinic keratosis.

Such compositions are useful in prevention, treatment and inhibition of benign and malignant tumors and neoplasia including neoplasia in metastasis, for example in colorectal cancer, brain cancer, bone cancer, epithelial cell-derived neoplasia (epithelial carcinoma) such as basal cell carcinoma, adenocarcinoma, gastrointestinal cancer such as lip cancer, mouth cancer, esophageal cancer, small bowel cancer, stomach cancer, colon cancer, liver cancer, bladder cancer, pancreas cancer, ovary cancer, cervical cancer, lung cancer, breast cancer, skin cancer such as squamous cell and basal cell cancers, prostate cancer, renal cell carcinoma, and other known cancers that effect epithelial cells throughout the body. Neoplasias for which compositions of the invention are contemplated to be particularly useful are gastrointestinal cancer, Barrett's esophagus, liver cancer, bladder cancer, pancreatic cancer, ovarian cancer, prostate cancer, cervical cancer, lung cancer, breast cancer and skin cancer. Such compositions can also be used to treat fibrosis that occurs with radiation therapy. Such compositions can be used to treat subjects having adenomatous polyps, including those with familial adenomatous polyposis (FAP). Additionally, such compositions can be used to prevent polyps from forming in patients at risk of FAP.

More particularly, the compositions can be used in treatment, prevention and inhibition of acral lentiginous melanoma, actinic keratoses, adenocarcinoma, adenoid cystic carcinoma, adenoma, adenosarcoma, adenosquamous carcinoma, astrocytic tumors, bartholin gland carcinoma, basal cell carcinoma, breast cancer, bronchial gland carcinoma, capillary hemangioma, carcinoids, carcinosarcoma, cavernous hemangioma, cholangiocarcinoma, chondrosarcoma, chorioid plexus papilloma or carcinoma, clear cell carcinoma, cutaneous T-cell lymphoma (mycosis fungoides), cystadenoma, displastic nevi, endodermal sinus tumor, endometrial hyperplasia, endometrial stromal sarcoma, endometrioid adenocarcinoma, ependymoma, epithelioid angiomatosis, Ewing's sarcoma, fibrolamellar sarcoma, focal nodular hyperplasia, gastrinoma, germ cell tumors, glioblastoma, glucagonoma, hemangioblastoma, hemangioendothelioma, hemangioma, hepatic adenoma, hepatic adenomatosis, hepatocellular carcinoma, insulinoma, intraepithelial neoplasia, interepithelial squamous cell neoplasia, invasive squamous cell carcinoma, Kaposi's sarcoma, large cell carcinoma, leiomyosarcoma, lentigo-maligna melanoma, malignant melanoma, malignant mesothelial tumors, medulloblastoma, medulloepithelioma, melanoma, meningioma, mesothelioma, mucoepidermoid carcinoma, neuroblastoma, neuroepithelial adenocarcinoma, nodular melanoma, oat cell carcinoma, oligodendroglioma, osteosarcoma, papillary serous adenocarcinoma, pineal tumors, pituitary tumors, plasmacytoma, pseudosarcoma, pulmonary blastoma, renal cell carcinoma, retinoblastoma, rhabdomyosarcoma, sarcoma, serous carcinoma, small cell carcinoma, soft tissue carcinoma, somatostatin-secreting tumor, squamous carcinoma, squamous cell carcinoma, submesothelial carcinoma, superficial spreading melanoma, undifferentiated carcinoma, uveal melanoma, verrucous carcinoma, vipoma, well differentiated carcinoma and Wilm's tumor.

Such compositions inhibit prostanoid-induced smooth muscle contraction by inhibiting synthesis of contractile prostanoids and hence can be of use in treatment of dysmenorrhea, premature labor, asthma and eosinophil-related disorders. They also can be of use for decreasing bone loss particularly in postmenopausal women (i.e., treatment of osteoporosis), and for treatment of glaucoma.

Preferred uses for compositions of the invention are for treatment of rheumatoid arthritis and osteoarthritis, for pain management generally (particularly post-oral surgery pain, post-general surgery pain, post-orthopedic surgery pain, and acute flares of osteoarthritis), for prevention and treatment of headache and migraine, for treatment of Alzheimer's disease, and for colon cancer chemoprevention.

Topical application of a composition of the invention can be especially useful in treatment of any kind of dermal disorder having an inflammatory component, whether malignant, non-malignant or pre-malignant, including scar formation and ketosis, and also including bums and solar damage, for example sunburn, wrinkles, etc. Such compositions can be used to treat inflammation resulting from a variety of skin injuries including without limitation those caused by viral diseases including herpes infections (e.g., cold sores, genital herpes), shingles and chicken pox. Other lesions or injuries to the skin that can be treated with such compositions include pressure sores (decubitus ulcers), hyperproliferative activity in the epidermis, miliria, psoriasis, eczema, acne, dermatitis, itching, warts and rosacea. Such compositions can also facilitate healing processes after surgical procedures, including cosmetic procedures such as chemical peels, laser treatment, dermabrasion, face lifts, eyelid surgery, etc.

Besides being useful for human treatment, compositions of the invention are also useful for veterinary treatment of companion animals, exotic animals, farm animals, and the like, particularly mammals including rodents. More particularly, compositions of the invention are useful for veterinary treatment of COX-2 mediated disorders in horses, dogs and cats.

The present compositions can be used in combination therapies with opioids and other analgesics, including narcotic analgesics, Mu receptor antagonists, Kappa receptor antagonists, non-narcotic (i.e. non-addictive) analgesics, monoamine uptake inhibitors, adenosine regulating agents, cannabinoid derivatives, Substance P antagonists, neurokinin-1 receptor antagonists and sodium channel blockers, among others. Preferred combination therapies comprise use of a composition of the invention with one or more compounds selected from aceclofenac, acemetacin, ε-acetamidocaproic acid, acetaminophen, acetaminosalol, acetanilide, acetylsalicylsalicylic acid, S-adenosylmethionine, alclofenac, alfentanil, allylprodine, alminoprofen, aloxiprin, alphaprodine, aluminum bis(acetylsalicylate), amfenac, aminochlorthenoxazin, 3-amino-4-hydroxybutyric acid, 2-amino-4-picoline, aminopropylon, aminopyrine, amixetrine, ammonium salicylate, ampiroxicam, amtolmetin guacil, anileridine, antipyrine, antipyrine salicylate, antrafenine, apazone, aspirin, balsalazide, bendazac, benorylate, benoxaprofen, benzpiperylon, benzydamine, benzylmorphine, berberine, bermoprofen, bezitramide, α-bisabolol, bromfenac, p-bromoacetanilide, 5-bromosalicylic acid acetate, bromosaligenin, bucetin, bucloxic acid, bucolome, bufexamac, bumadizon, buprenorphine, butacetin, butibufen, butorphanol, calcium acetylsalicylate, carbamazepine, carbiphene, carprofen, carsalam, chlorobutanol, chlorthenoxazin, choline salicylate, cinchophen, cinmetacin, ciramadol, clidanac, clometacin, clonitazene, clonixin, clopirac, clove, codeine, codeine methyl bromide, codeine phosphate, codeine sulfate, cropropamide, crotethamide, desomorphine, dexoxadrol, dextromoramide, dezocine, diampromide, diclofenac, difenamizole, difenpiramide, diflunisal, dihydrocodeine, dihydrocodeinone enol acetate, dihydromorphine, dihydroxyaluminum acetylsalicylate, dimenoxadol, dimepheptanol, dimethylthiambutene, dioxaphetyl butyrate, dipipanone, dipyrocetyl, dipyrone, ditazol, droxicam, emorfazone, enfenamic acid, epirizole, eptazocine, etanercept, etersalate, ethenzamide, ethoheptazine, ethoxazene, ethylmethylthiambutene, ethylmorphine, etodolac, etofenamate, etonitazene, eugenol, felbinac, fenbufen, fenclozic acid, fendosal, fenoprofen, fentanyl, fentiazac, fepradinol, feprazone, floctafenine, flufenamic acid, flunoxaprofen, fluoresone, flupirtine, fluproquazone, flurbiprofen, fosfosal, gentisic acid, glafenine, glucametacin, glycol salicylate, guaiazulene, hydrocodone, hydromorphone, hydroxypethidine, ibufenac, ibuprofen, ibuproxam, imidazole salicylate, indomethacin, indoprofen, infliximab, interleukin-10, isofezolac, isoladol, isomethadone, isonixin, isoxepac, isoxicam, ketobemidone, ketoprofen, ketorolac, p-lactophenetide, lefetamine, levorphanol, lexipafant, lofentanil, lonazolac, lornoxicam, loxoprofen, lysine acetylsalicylate, magnesium acetylsalicylate, meclofenamic acid, mefenamic acid, meperidine, meptazinol, mesalamine, metazocine, methadone, methotrimeprazine, metiazinic acid, metofoline, metopon, mofebutazone, mofezolac, morazone, morphine, morphine hydrochloride, morphine sulfate, morpholine salicylate, myrophine, nabumetone, nalbuphine, 1-naphthyl salicylate, naproxen, narceine, nefopam, nicomorphine, nifenazone, niflumic acid, nimesulide, 5′-nitro-2′-propoxyacetanilide, norlevorphanol, normethadone, normorphine, norpipanone, olsalazine, opium, oxaceprol, oxametacine, oxaprozin, oxycodone, oxymorphone, oxyphenbutazone, papaveretum, paranyline, parsalmide, pentazocine, perisoxal, phenacetin, phenadoxone, phenazocine, phenazopyridine hydrochloride, phenocoll, phenoperidine, phenopyrazone, phenyl acetylsalicylate, phenylbutazone, phenyl salicylate, phenyramidol, piketoprofen, piminodine, pipebuzone, piperylone, pirazolac, piritramide, piroxicam, pirprofen, pranoprofen, proglumetacin, proheptazine, promedol, propacetamol, propiram, propoxyphene, propyphenazone, proquazone, protizinic acid, ramifenazone, remifentanil, rimazolium metilsulfate, salacetamide, salicin, salicylamide, salicylamide o-acetic acid, salicylsulfuric acid, salsalate, salverine, simetride, sodium salicylate, sufentanil, sulfasalazine, sulindac, superoxide dismutase, suprofen, suxibuzone, talniflumate, tenidap, tenoxicam, terofenamate, tetrandrine, thiazolinobutazone, tiaprofenic acid, tiaramide, tilidine, tinoridine, tolfenamic acid, tolmetin, tramadol, tropesin, viminol, xenbucin, ximoprofen, zaltoprofen, ziconotide and zomepirac (see The Merck Index, 13th Edition (2001), Therapeutic Category and Biological Activity Index, lists therein headed “Analgesic”, “Anti-inflammatory” and “Antipyretic”).

Particularly preferred combination therapies comprise use of a composition of the invention, for example a celecoxib or valdecoxib composition of the invention, with an opioid compound, more particularly where the opioid compound is codeine, meperidine, morphine or a derivative thereof.

The compound to be administered in combination with the selective COX-2 inhibitory drug can be formulated separately therefrom, and administered by any suitable route, including orally, rectally, parenterally or topically to the skin or elsewhere. Alternatively, the compound to be administered in combination with the selective COX-2 inhibitory drug can be coformulated therewith in a dermally deliverable composition of the invention.

In an embodiment of the invention, particularly where the COX-2 mediated condition is headache or migraine, the present selective COX-2 inhibitory drug composition is administered in combination therapy with a vasomodulator, preferably a xanthine derivative having vasomodulatory effect, more preferably an alkylxanthine compound.

Combination therapies wherein an alkylxanthine compound is co-administered with a selective COX-2 inhibitory drug composition as provided herein are embraced by the present embodiment of the invention whether or not the alkylxanthine is a vasomodulator and whether or not the therapeutic effectiveness of the combination is to any degree attributable to a vasomodulatory effect. The term “alkylxanthine” herein embraces xanthine derivatives having one or more C₁₋₄ alkyl, preferably methyl, substituents, and pharmaceutically acceptable salts of such xanthine derivatives. Dimethylxanthines and trimethylxanthines, including caffeine, theobromine and theophylline, are especially preferred. Most preferably, the alkylxanthine compound is caffeine.

The total and relative dosage amounts of the selective COX-2 inhibitory drug and of the vasomodulator or alkylxanthine are selected to be therapeutically and/or prophylactically effective for relief of pain associated with the headache or migraine. Suitable dosage amounts will depend on the particular selective COX-2 inhibitory drug and the particular vasomodulator or alkylxanthine selected. For example, in a combination therapy with celecoxib and caffeine, typically the celecoxib will be administered in a daily dosage amount of about 50 mg to about 1000 mg, preferably about 100 mg to about 600 mg, and the caffeine in a daily dosage amount of about 1 mg to about 500 mg, preferably about 10 mg to about 400 mg, more preferably about 20 mg to about 300 mg.

The vasomodulator or alkylxanthine component of the combination therapy can be administered in any suitable dosage form by any suitable route, including orally, rectally, parenterally or topically to the skin or elsewhere. The vasomodulator or alkylxanthine can optionally be coformulated with the selective COX-2 inhibitory drug in a single transdermal dosage form. Thus a transdermal composition of the invention optionally comprises both a selective COX-2 inhibitory drug and a vasomodulator or alkylxanthine such as caffeine, in total and relative amounts consistent with the dosage amounts set out hereinabove.

The phrase “in total and relative amounts effective to relieve pain”, with respect to amounts of a selective COX-2 inhibitory drug and a vasomodulator or alkylxanthine in a composition of the present embodiment, means that these amounts are such that (a) together these components are effective to relieve pain, and (b) each component is or would be capable of contribution to a pain-relieving effect if the other component is or were not present in so great an amount as to obviate such contribution.

### EXAMPLES

This invention will be more fully described by way of the following Examples but is not limited to these Examples. The term “parecoxib” is used in these Examples in the strict sense of parecoxib acid except where otherwise indicated; for example “parecoxib Na” means the sodium salt of parecoxib.

As a way of measuring the skin permeation properties of selective COX-2 inhibitory drugs or prodrugs in dermally deliverable pharmaceutical compositions, a Franz diffusion cell was provided utilizing a human cadaver skin membrane and a 1% polysorbate 80 (Tween™ 80) solution as a receptor fluid. Frozen skin was thawed at room temperature and punched with a 20 mm puncher to provide a membrane. The receptor compartment of the Franz diffusion cell was filled with the receptor fluid and the diffusion cell was maintained at 32° C. The membrane was mounted on the receptor compartment, covered and fastened with a clamp. Air bubbles were removed from the receptor fluid, which was allowed to equilibrate for 30 minutes. A test composition was brought into contact with the membrane. The amount of drug which permeated through the membrane in a 24 hour period was determined by HPLC analysis of the receptor fluid. Each test was conducted in several replicates.

### Example 1

Saturated solutions of celecoxib were prepared in the following solvents: 70% aqueous ethanol (EtOH), ethanol, PEG-400 and propylene glycol (PG). The solutions were tested for skin permeation properties as described above, using 250 μl drops of each test solution. Results are shown in Table 1.

### Example 2

Saturated solutions of valdecoxib were prepared and tested exactly as described for the celecoxib solutions in Example 1. Results are shown in Table 1.

TABLE 1
Skin flux from saturated celecoxib and valdecoxib solutions

| Drug | Celecoxib | | | | Valdecoxib | | | |
|---|---|---|---|---|---|---|---|---|
| Solvent | 70% | EtOH | PEG- | PG | 70% | EtOH | PEG- | PG |
| | EtOH | | 400 | | EtOH | | 400 | |
| Concentration | 15.2 | 91.4 | 297 | 33.3 | 12.7 | 7.48 | 210 | 23.6 |
| (mg/ml) | | | | | | | | |
| Skin flux | 15.7 ± 3.83 | 5.62 ± 1.49 | ud | ud | 12.8 ± 4.96 | 1.44 ± 0.54 | ud | ud |
| (μg/cm² · day) | | | | | | | | |

ud = undetectable

No skin permeation of either celecoxib or valdecoxib was observed over a 24 hour period when PEG-400 or propylene glycol was used as the solvent. Surprisingly, 70% aqueous ethanol provided greater skin flux of both celecoxib and valdecoxib than ethanol alone. With this solvent, skin permeation rates of celecoxib and valdecoxib were similar (15.7 and 12.8 μg/cm².day respectively).

### Example 3

A saturated solution of parecoxib sodium in 70% aqueous ethanol was prepared and tested exactly as described for the celecoxib and valdecoxib solutions in Examples 1 and 2. Since different lots of skin were used to determine the skin flux of each compound, a standard was run on each skin lot and the data were normalized. Results, together with the corresponding celecoxib and valdecoxib results from above, are shown in Table 2.

TABLE 2
Skin flux from saturated solutions in 70% aqueous ethanol

| Drug or | Concentration | Skin flux | Normalized skin flux |
|---|---|---|---|
| prodrug | (mg/ml) | (μg/cm² · day) | (μg/cm² · day) |
| celecoxib | 15.2 | 15.7 ± 3.83 | 34.9 |
| valdecoxib | 12.7 | 12.8 ± 4.96 | 53.4 |
| parecoxib Na | 386 | 254 ± 164 | 120.0 |

### Example 4

To saturated solutions of celecoxib, valdecoxib and parecoxib sodium in 70% aqueous ethanol prepared as in Example 3 were added 5% oleyl alcohol and 3% thymol by weight as permeation enhancers were prepared for celecoxib, valdecoxib and parecoxib sodium. The solutions were tested for skin permeation properties as described above, using 250 μl drops of each test solution. An enhancement factor was calculated by comparison with the skin flux data in Table 2 above. Results are shown in Table 3.

TABLE 3
Skin flux from saturated solutions in 70% aqueous ethanol containing
5% oleyl alcohol and 3% thymol

| Drug or prodrug | Skin flux (μg/cm² · day) | Enhancement factor |
|---|---|---|
| celecoxib | 21.7 ± 4.6 | 1.4 |
| valdecoxib | 323 ± 21 | 25 |
| parecoxib Na | 1210 ± 58.0 | 4.8 |

The combination of oleyl alcohol and thymol gave an especially pronounced enhancement of skin flux in the case of valdecoxib.

### Example 5

Saturated solutions of valdecoxib (5-1,5-2 and 5-3) were prepared using various solvents and permeation enhancers as carriers. The solutions were tested for skin permeation properties as described above. Carrier compositions are shown in Table 4 and valdecoxib concentration and skin flux data in Table 5.

TABLE 4
Carrier compositions (% by weight)

| Composition | 5-1 | 5-2 | 5-3 |
|---|---|---|---|
| water | 30 | 33 | 30 |
| ethanol | 62 | 62 | 30 |
| isopropanol | — | — | 10 |
| 1,3-butanediol | — | — | 22 |
| oleyl alcohol | 5 | 5 | 5 |
| thymol | 3 | — | 3 |

TABLE 5
Valdecoxib concentration and skin flux

| Composition | 5-1 | 5-2 | 5-3 |
|---|---|---|---|
| Concentration (mg/ml) | 22.0 | 18.5 | 13.4 |
| Skin flux (μg/cm² · day) | 441 ± 160 | 287 ± 23.9 | 302 ± 48.9 |

### Example 6

Gel compositions of celecoxib and valdecoxib (each 1% by weight) were prepared as solutions in 70% aqueous ethanol, together with 3% by weight Klucel™ (hydroxypropylcellulose) as a thickening agent. These were non-occlusively tested, using a 50 mg amount of each gel, for skin permeation as described above. Distribution of drug in epidermis and dermis was also determined. Results are shown in Table 6, by comparison with the solution compositions of Examples 1 and 2.

TABLE 6
Skin flux from solution and gel compositions

| Drug | Celecoxib | | Valdecoxib | |
|---|---|---|---|---|
| Formulation | Solution | Gel | Solution | Gel |
| Concentra- | 15.2 | 10 | 12.7 | 10 |
| tion (mg/ml) | | | | |
| Amount | 250 μl | 50 mg | 250 μl | 50 mg |
| applied | | | | |
| Occlusive? | yes | no | yes | no |
| Skin flux | 15.7 ± 3.83 | 3.82 ± 3.36 | 12.8 ± 4.96 | 11.3 ± 6.48 |
| (μg/ | | | | |
| cm² · day) | | | | |
| Drug in epi- | 3.92 ± 0.79 | 2.36 ± 1.06 | 9.27 ± 3.84 | 1.81 ± 1.87 |
| dermis (μg) | | | | |
| Drug in | 2.50 ± 1.53 | 1.22 ± 0.51 | 0.543 ± 0.525 | ud |
| dermis (μg) | | | | |

ud = undetectable

### Example 7

In a 67% aqueous ethanol solvent containing 5% parecoxib sodium, saturated solutions of celecoxib and valdecoxib were prepared. Skin fluxes of both parecoxib sodium and either celecoxib or valdecoxib were determined as described above. Enhancement factors for celecoxib and valdecoxib skin flux, by comparison with the data in Table 2 above in absence of parecoxib sodium, were calculated. Results are shown in Table 7.

TABLE 7
Skin flux from combination compositions of parecoxib and either
celecoxib or valdecoxib

| Drug | Celecoxib + parecoxib Na | | Valdecoxib + parecoxib Na | |
|---|---|---|---|---|
| composition | celecoxib | parecoxib | valdecoxib | parecoxib |
| Concentration | 15.9 | 49.4 | 19.2 | 49.7 |
| (mg/ml) | | | | |
| Skin flux (μg/ | 183 ± 153 | 74.7 ± 14.7 | 108 ± 16.7 | 64.1 ± 11.3 |
| cm² · day) | | | | |
| Enhancement | 11.5 | | 8.4 | |
| factor | | | | |

Surprisingly, the presence of parecoxib sodium in the solution greatly enhanced skin permeation of both celecoxib and valdecoxib.

### Example 8

Gel formulations (Compositions 8-1 to 8-3) containing 2.5% or 5% celecoxib were prepared as solutions in 70% aqueous ethanol, together with 2% hydroxypropylcellulose (Klucel™) and 1% polysorbate 80 (Tween™ 80).

Composition 8-1 contained no HPMC, and Compositions 8-2 and 8-3 contained 3% HPMC (Methocel™ E15LV). The gels were tested for skin permeation properties as described in Example 6. Skin permeation results are shown in Table 8.

TABLE 8
Skin flux from celecoxib gel compositions

| | | | | Skin flux (μg/ |
|---|---|---|---|---|
| Composition | Celecoxib (%) | HPMC (%) | Replicates | cm² · day) |
| 8-1 | 2.5 | 0 | 7 | 5.64 ± 3.38 |
| 8-2 | 2.5 | 3 | 6 | 9.34 ± 4.70 |
| 8-3 | 5 | 3 | 8 | 8.90 ± 5.57 |

Example 9

Gel formulations containing 2.5% celecoxib were prepared as in Example 8, but with further addition of 0.5% carbomer and 0.4% 2-amino-2-methyl-1-propanol (AMP-95™) and with various grades of HPMC incorporated at 3%. The gels were tested as described in Example 6, by comparison with a saturated celecoxib solution in 70% aqueous ethanol and a celecoxib gel (Composition 8-1) prepared as above with no HPMC. The average amount of celecoxib found in receptor fluid after 15 hours is presented in Table 9.

TABLE 9
Skin permeation in 15 hours from celecoxib gel compositions

| | | Celecoxib |
|---|---|---|
| Composition | HPMC | permeation (μg/cm²) |
| saturated in 70% ethanol | | 1.406 ± 0.086 |
| 8-1 | none | 1.464 ± 0.246 |
| 9-1 | 3% Methocel ™ F4M | 1.821 ± 0.452 |
| 9-2 | 3% Methocel ™ E50LV | 2.511 ± 0.959 |
| 9-3 | 3% Methocel ™ E15LV | 1.900 ± 0.260 |

### Example 10

Saturated aqueous solutions of celecoxib, valdecoxib and parecoxib were prepared and skin flux was determined at various temperatures, as described in previous examples, using 3 replicates. Results are presented in Table 10.

TABLE 10
Skin flux at different temperatures

| | | Skin flux (μg/cm² · day) | |
|---|---|---|---|
| Compound | Concentration (μg/ml) | 32° C. | 50° C. |
| celecoxib | 0.5 | 4.27 ± 0.84 | 23.71 ± 4.42 |
| valdecoxib | 12.1 | 7.94 ± 0.89 | 42.12 ± 7.82 |
| parecoxib | 50.8 | 8.62 ± 1.94 | 47.16 ± 3.70 |

### Example 11

Gel formulations (Compositions 11-1 and 11-2) containing 2% parecoxib sodium and excipient ingredients as shown in Table 11 were prepared as follows. Tween™ 80 (polysorbate 80) was mixed with water in a first container. HPMC 2910 was added slowly to the resulting aqueous mixture until it was completely dispersed. Ethanol, parecoxib sodium, propylene glycol, thymol and oleyl alcohol were mixed in a second container. The resulting mixture was added to the aqueous mixture in the first container and mixed well. Klucel™ (hydroxypropylcellulose) was added slowly with further mixing.

It will be seen that Compositions 11-1 and 11-2 differ in the amount of propylene glycol they contain.

TABLE 11
Composition (% by weight) of gel formulations

| Composition | 11-1 | 11-2 |
|---|---|---|
| parecoxib Na | 2 | 2 |
| hydroxypropylcellulose | 3 | 3 |
| HPMC 2910 | 3 | 3 |
| polysorbate 80 | 1 | 1 |
| propylene glycol | 10 | 20 |
| thymol | 2 | 2 |
| oleyl alcohol | 5 | 5 |
| ethanol | 50 | 40 |
| water | 24 | 24 |

Compositions 11-1 and 11-2 were tested non-occusively for skin permeation properties as described in previous examples, using 3 replicates. Both compositions were tested in a volume of 100 μl; Composition 11-1 was additionally tested in volumes of 50 and 20 μl. Skin flux was recorded as shown in Table 12.

TABLE 12
Skin flux for gel formulations of parecoxib sodium

| Composition | Volume (μl) | Skin flux (μg/cm² · day) |
|---|---|---|
| 11-2 | 100 | 27.3 ± 7.0 |
| 11-1 | 100 | 27.7 ± 7.6 |
| 11-1 | 50 | 21.1 ± 11.6 |
| 11-1 | 20 | 3.6 ± 2.4 |

### Example 12

Liquid formulations (Compositions 12-1 and 12-2) were prepared as simple solutions. Composition 12-1 contained 1% celecoxib, 30% water, and 69% ethanol, by weight. Composition 12-2 contained 1% celecoxib, 30% water, 59% ethanol and 10% urea, by weight. Both compositions were tested occlusively for skin flux in a volume of 500 μl. Results are shown in Table 13.

TABLE 13
Skin flux for liquid formulations of celecoxib

| Composition | Replicates | Skin flux (μg/cm² · day) |
|---|---|---|
| 13-1 | 1 | 2.02 |
| 13-2 | 3 | 5.41 ± 3.45 |

### Example 13

Gel formulations (Compositions 13-1 to 13-4) containing 2% parecoxib sodium and excipient ingredients as shown in Table 14 were prepared by the procedure described in Example 11.

TABLE 14
Composition (% by weight) of gel formulations

| Composition | 14-1 | 14-2 | 14-3 | 14-4 |
|---|---|---|---|---|
| parecoxib Na | 2 | 2 | 2 | 2 |
| hydroxypropylcellulose | 3 | 3 | 3 | 3 |
| HPMC 2910 | 0 | 3 | 0 | 3 |
| polysorbate 80 | 0 | 0 | 1 | 1 |
| Oleyl alcohol | 5 | 5 | 5 | 5 |
| thymol | 2 | 2 | 2 | 2 |
| propylene glycol | 10 | 11 | 11 | 11 |
| ethanol | 50 | 43 | 44 | 42 |
| water | 28 | 31 | 32 | 31 |

Compositions 13-1 to 13-4 were tested for skin permeation properties as described in previous examples, using 3 replicates. Formulations were tested non-occlusively in a volume of 50 μl. Skin flux data are shown in Table 15.

TABLE 15
Skin flux for gel formulations of parecoxib sodium

| Composition | Skin flux (μg/cm² · day) |
|---|---|
| 13-1 | 8.92 ± 8.52 |
| 13-2 | 6.73 ± 6.72 |
| 13-3 | 20.67 ± 7.48 |
| 13-4 | 21.11 ± 11.62 |

### Example 14

Saturated solutions of parecoxib acid (Compositions 14-1 to 14-4) were prepared as shown in Table 16. After addition of parecoxib the solutions were mixed for 3 hours on a rotating mixer.

TABLE 16
Composition (% by weight) of parecoxib acid formulations

| Composition | 14-1 | 14-2 | 14-3 | 14-4 |
|---|---|---|---|---|
| ethanol | 7.0 | 6.5 | 6.5 | 6.5 |
| water | 3.0 | 3.0 | 3.0 | 3.0 |
| parecoxib acid | saturated | saturated | saturated | saturated |
| lauryl lactate | | 0.5 | | |
| myristyl lactate | | | 0.5 | |
| glyceryl dilaurate | | | | saturated |

Compositions 14-1 to 14-4 were tested for skin permeation properties as described in previous examples, in a volume of 300 μl, using 3 replicates. Skin flux data are shown in Table 17.

TABLE 17
Skin flux for parecoxib acid solutions

| Composition | Skin flux (μg/cm² · day) |
|---|---|
| 14-1 | 33.9 ± 19.68 |
| 14-2 | 104.4 ± 15.36 |
| 14-3 | 167.0 ± 44.4 |
| 14-4 | 86.2 ± 15.6 |

### Example 15

Gel formulations (Compositions 15-1 to 15-4) containing 2% parecoxib sodium and excipient ingredients as shown in Table 18 were prepared by the procedure described in Example 11.

TABLE 18
Composition (% by weight) of gel formulations

| | | | | |
|---|---|---|---|---|
| Composition | 15-1 | 15-2 | 15-3 | 15-4 |

| | | | | |
|---|---|---|---|---|
| parecoxib Na | 2 | 2 | 2 | 2 |
| hydroxypropylcellulose | 3 | 3 | 3 | 3 |
| HPMC 2910 | 3 | 3 | 3 | 3 |
| polysorbate 80 | 1 | 1 | 1 | 1 |
| oleyl alcohol | 5 | 5 | 5 | 5 |
| thymol | 2 | 2 | 2 | 2 |
| lauryl lactate | 2 | 2.5 | 3 | 0 |
| myristyl lactate | 2 | 2.5 | 0 | 3 |
| glyceryl dilaurate | 1 | 0 | 2 | 2 |
| propylene glycol | 10 | 10 | 10 | 10 |
| ethanol | 40 | 40 | 40 | 40 |
| water | 29 | 29 | 29 | 29 |

Compositions 15-1 to 15-4 were tested for skin permeation properties as described in previous examples, using 3 replicates. Formulations were tested non-occlusively in a volume of 50 μl. Skin flux data are shown in Table 19.

TABLE 19
Skin flux for gel formulations of parecoxib sodium

| Composition | Skin flux (μg/cm² · day) |
|---|---|
| 15-1 | 67.7 ± 47.4 |
| 15-2 | 31.6 ± 4.0 |
| 15-3 | 55.3 ± 34.2 |
| 15-4 | 39.0 ± 3.1 |

### Example 16

Gel formulations (Compositions 16-1 to 16-13) containing 2% celecoxib, 2% parecoxib or 2% parecoxib sodium, in each case with excipient ingredients as shown in Tables 20A and 20B, were prepared by the procedure described in Example 11.

TABLE 20A
Composition (% by weight) of gel formulations

| Composition | 16-1 | 16-2 | 16-3 | 16-4 | 16-5 | 16-6 | 16-7 |
|---|---|---|---|---|---|---|---|
| celecoxib | 2 | 2 | 2 | | | | |
| parecoxib | | | | | | 2 | 2 |
| parecoxib Na | | | | 2 | 2 | | |
| carbomer 980 | 0.5 | 0.5 | | 0.5 | 0.5 | 0.5 | 0.5 |
| hydroxypropyl- | | | | | | | |
| cellulose | | | | | | | |
| HPMC 2910 | 3 | 3 | 6 | 3 | 3 | 3 | 3 |
| polysorbate 80 | 1 | 1 | 1 | 1 | 1 | 1 | 1 |
| 2-amino-2-methyl-1- | 0.4 | 0.4 | | 0.2 | 0.4 | 0.4 | 0.4 |
| propanol | | | | | | | |
| thymol | | 3 | 3 | | 3 | | 3 |
| oleyl alcohol | | 5 | 2.5 | | 5 | | 5 |
| glyceryl oleate | | 5 | 2.5 | | 5 | | |
| ethanol | 65 | 58 | 50 | 65 | 58 | 65 | 60 |
| water | 28.1 | 22.1 | 33 | 28.1 | 22.1 | 28.1 | 25.1 |
| pH | 7.50 | 8.45 | | | | | |

TABLE 20B
Composition (% by weight) of gel formulations

| Composition | 16-8 | 16-9 | 16-10 | 16-11 | 16-12 | 16-13 |
|---|---|---|---|---|---|---|
| celecoxib | | | | | | |
| parecoxib | 2 | 2 | 2 | 2 | 2 | 2 |
| parecoxib Na | | | | | | |
| carbomer 980 | 0.5 | | | | | |
| hydroxypropyl- | | | | 3 | 3 | 3 |
| cellulose | | | | | | |
| HPMC 2910 | 3 | 6 | 6 | 2 | 2 | 2 |
| polysorbate 80 | 1 | 2 | 1 | 1 | 1 | 1 |
| 2-amino-2-methyl- | 0.4 | | | | | |
| 1-propanol | | | | | | |
| thymol | 3 | | 3 | | 2 | 2 |
| oleyl alcohol | 5 | | 2.5 | | 5 | 5 |
| glyceryl oleate | | | 2.5 | | | 5 |
| ethanol | 58 | 65 | 50 | 65 | 62 | 62 |
| water | 22.1 | 25 | 33 | 27 | 23 | 18 |
| pH | | 4.40 | | 4.44 | 4.71 | 4.31 |

Compositions 16-1, 16-2, 16-9 and 16-11 to 16-13 were tested for skin permeation properties as described in previous examples, using 3 replicates. Formulations were tested non-occlusively in a volume of 100 μl. Skin flux data are shown in Table 21.

TABLE 21
Skin flux for gel formulations

| Composition | Skin flux (μg/cm² · day) |
|---|---|
| 16-1 | 5.51 ± 2.28 |
| 16-2 | 2.56 ± 0.69 |
| 16-9 | 14.0 ± 6.5 |
| 16-11 | 10.1 ± 1.4 |
| 16-12 | 80.4 ± 15.1 |
| 16-13 | 74.7 ± 17.1 |

### Example 17

Saturated solutions of celecoxib (Compositions 17-1 to 17-6) were prepared as in previous examples, in the solvent systems shown in Table 22.

TABLE 22
Composition (% by weight) of solvent
systems for celecoxib solutions

| Composition | 17-1 | 17-2 | 17-3 | 17-4 | 17-5 | 17-6 |
|---|---|---|---|---|---|---|
| ethanol | 70 | 65 | 65 | 62 | 68 | 65 |
| water | 30 | 30 | 30 | 30 | 30 | 30 |
| glyceryl oleate¹ | | 5 | | | | |
| salicylic acid | | | 5 | | | |
| oleyl alcohol | | | | 5 | | |
| thymol | | | | 3 | | |
| sodium lauryl sulfate | | | | | 2 | |
| acetone | | | | | | 5 |

¹Arlacel ™ 186

Compositions 17-1 to 17-6 were tested for skin permeation properties as described in previous examples, in a volume of 300 μl, using 3 replicates. Skin flux data are shown in Table 23.

TABLE 23
Skin flux for saturated celecoxib solutions

| Composition | Skin flux (μg/cm² · day) |
|---|---|
| 17-1 | 2.60 ± 2.02 |
| 17-2 | 18.21 ± 11.04 |
| 17-3 | 6.02 ± 2.86 |
| 17-4 | 14.16 ± 0.48 |
| 17-5 | 4.05 ± 1.29 |
| 17-6 | 4.99 ± 1.03 |

### Example 18

A 1% celecoxib gel formulation was prepared having the composition shown in Table 24. In a first container, water and polysorbate 80 were mixed, and HPMC was then added until the HPMC was completely dispersed. In a second container, ethanol, celecoxib, propylene glycol and eucalyptus oil were mixed. The resulting mixture was poured into the mixture in the first container and mixed well. Finally, hydroxypropylcellulose was added slowly with mixing to form a gel.

TABLE 24
Composition (% by weight) of celecoxib gel formulation

| | |
|---|---|
| celecoxib | 1.0 |
| hydroxypropylcellulose | 3.0 |
| HPMC 2910 | 3.0 |
| polysorbate 80 | 1.0 |
| propylene glycol | 10.0 |
| eucalyptus oil | 0.2 |
| ethanol | 56.8 |
| water | 25.0 |

The composition was tested for skin permeation properties as described in previous examples. The gel formulation was tested non-occlusively in a volume of 100 μl. A skin flux of 7.58±19 μg/cm².day was determined for the 1% celecoxib gel formulation.

### Example 19

Saturated solutions of celecoxib and of valdecoxib were prepared in 70% aqueous ethanol. The solutions were tested for skin permeation properties as described in previous examples, using skin from different donors 1-4 and 6. The effect of skin donor on skin flux of celecoxib and valdecoxib from these solutions is shown in Table 25.

TABLE 25
Effect of skin donor on skin flux of celecoxib and valdecoxib

| | Skin flux (μg/cm² · day) | |
|---|---|---|
| Skin donor | celecoxib | valdecoxib |
| 1 | 15.7 ± 3.8 | |
| 2 | | 12.8 ± 5.0 |
| 3 | 73.7 ± 11.8 | 91.9 ± 15.0 |
| 4 | 31.9 ± 9.6 | 58.3 ± 11.0 |
| 6 | 50.4 ± 12.7 | |

### Example 20

A prototype parecoxib sodium gel formulation was prepared by methods composition as shown in Table 26.

TABLE 26
Prototype parecoxib sodium gel formulation

| | Prototype gel |
|---|---|
| Ingredient | (% by weight) |
| parecoxib Na | 2 |
| hydroxypropylcellulose | 3 |
| thymol | 1 |
| oleyl alcohol | 3 |
| myristyl lactate | 2 |
| lauryl lactate | 2.5 |
| glyceryl dilaurate | 0.5 |
| butylene glycol | 6 |
| propylene glycol | 4 |
| ethanol | 46 |
| water | 30 |

What is claimed is:

1. A dermally deliverable pharmaceutical composition comprising a therapeutic agent in a therapeutically effective amount solubilized in a solubilizing amount of a pharmaceutically acceptable carrier that comprises a low molecular weight monohydric alcohol, wherein (a) the therapeutic agent comprises at least one selective COX-2 inhibitory drug or prodrug thereof, and (b) a test sample of the composition provides a skin permeation rate of the therapeutic agent at least equal to that provided by a reference solution of the therapeutic agent in 70% aqueous ethanol.

2. The composition of claim 1 wherein substantially all of the therapeutic agent present is in solubilized form.

3. The composition of claim 1 wherein the therapeutic agent comprises at least one compound having the formula 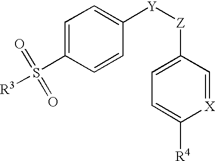
where R³ is a methyl, amino or imide group, R⁴ is hydrogen or a C₁₋₄ alkyl or alkoxy group, X is N or CR⁵ where R⁵ is hydrogen or halogen, and Y and Z are independently carbon or nitrogen atoms defining adjacent atoms of a five- to six-membered ring that is unsubstituted or substituted at one or more positions with oxo, halo, methyl or halomethyl groups; or an isomer, tautomer, pharmaceutically-acceptable salt or prodrug thereof.

4. The composition of claim 1 wherein the at least one selective COX-2 inhibitory drug or prodrug is selected from the group consisting of celecoxib, deracoxib, valdecoxib, parecoxib, rofecoxib, etoricoxib, 2-(3,5-difluorophenyl)-3-[4-(methylsulfonyl)phenyl]-2-cyclopenten-1-one, (S)-6,8-dichloro-2-(trifluoromethyl)-2H-1-benzopyran-3-carboxylic acid, 2-(3,4-difluorophenyl)-4-(3-hydroxy-3-methyl-11-butoxy)-5-[4-(methylsulfonyl)phenyl]-3-(2H)-pyridazinone, 5-methyl-2-(2′-chloro-6′-fluoroanilino)phenylacetic acid and salts thereof.

5. The composition of claim 1 wherein the at least one selective COX-2 inhibitory drug or prodrug is selected from the group consisting of celecoxib, valdecoxib, parecoxib and its salts, rofecoxib and etoricoxib.

6. The composition of claim 1 wherein the at least one selective COX-2 inhibitory drug or prodrug is valdecoxib or a prodrug thereof.

7. The composition of claim 1 wherein the at least one selective COX-2 inhibitory drug or prodrug is parecoxib or a salt thereof.

8. The composition of claim 1 wherein the monohydric alcohol is a C₂₋₆ monohydric alcohol.

9. The composition of claim 8 wherein the C₂₋₆ monohydric alcohol is selected from the group consisting of ethanol, isopropanol, n-butanol and diethylene glycol monoethyl ether.

10. The composition of claim 1 that is in a liquid or semi-solid dosage form.

11. The composition of claim 10 in a dosage form selected from the group consisting of creams, pastes, gels, ointments, lotions and aerosols.

12. The composition of claim 1 exhibiting a skin permeation rate of the therapeutic agent not less than about 10 μg/cm².day.

13. The composition of claim 1 exhibiting a skin permeation rate of the therapeutic agent not less than about 25 μg/cm².day.

14. The composition of claim 1, further comprising at least one skin permeation enhancer.

15. The composition of claim 14 wherein the at least one skin permeation enhancer is selected from the group consisting of terpenes, terpenoids, fatty alcohols and derivatives thereof, fatty acids and alkyl and glyceryl esters thereof, fatty acid esters of glycolic acid and its salts, lactate esters of fatty alcohols, laurocapram and derivatives thereof, dimethylsulfoxide, n-decyl methylsulfoxide, salicylic acid and alkyl esters thereof, N,N-dimethylacetamide, dimethylformamide, N,N-dimethyltoluamide, 2-pyrrolidinone and N-alkyl derivatives thereof, 2-nonyl-1,3-dioxolane, eucalyptol, sorbitan esters and sunscreens.

16. The composition of claim 14 wherein the at least one skin permeation enhancer is selected from the group consisting of oleyl alcohol, methyl salicylate, NMP, thymol, menthol, carvone, carveol, citral, dihydrocarveol, dihydrocarvone, neomenthol, isopulegol, 4-terpinenol, menthone, pulegol, camphor, geraniol, α-terpineol, linalool, carvacrol, trans-anethole, isomers thereof and racemic mixtures thereof.

17. The composition of claim 14 that comprises a fatty alcohol and a terpene or terpenoid as skin permeation enhancers.

18. The composition of claim 14 that comprises oleyl alcohol and thymol as skin permeation enhancers.

19. The composition of claim 14 wherein the at least one skin permeation enhancer is selected from the group consisting of oleic acid, isopropyl laurate, isopropyl myristate, methyl oleate, glyceryl monolaurate, glyceryl monooleate, glyceryl dilaurate, glyceryl dioleate, lauroyl glycolate, caproyl glycolate, cocoyl glycolate, isostearoyl glycolate, sodium lauroyl glycolate, tromethamine lauroyl glycolate, lauryl lactate, myristyl lactate and oleyl lactate.

20. The composition of claim 14 wherein the at least one skin permeation enhancer is glyceryl monolaurate.

21. The composition of claim 14 wherein the at least one skin permeation enhancer is a compound of formula 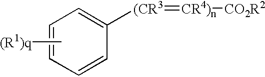
where R¹ groups are independently hydrogen, lower alkyl, lower alkoxy, halogen, hydroxyl or NR⁵R⁶ groups in which R⁵ and R⁶ are independently hydrogen or lower alkyl groups or R⁵ and R⁶ together with the nitrogen atom to which they are attached form a 5- or 6-membered heterocyclic ring; R² is a C₅₋₁₈ linear, branched or cyclic alkyl group; R³ is a hydrogen or phenyl group; R⁴ is a hydrogen or cyano group; n is 0 or 1; and q is 1 or 2.

22. The composition of claim 14 wherein the at least one skin permeation enhancer is selected from the group consisting of C₅₋₁₈ alkyl esters of p-aminobenzoic acid (PABA), p-dimethylaminobenzoic acid, 2-aminobenzoic acid, cinnamic acid, p-methoxycinnamic acid, salicylic acid and 2-cyano-3,3-diphenylacrylic acid.

23. The composition of claim 1 wherein the therapeutic agent is present at a concentration in the composition of about 12.5 to about 400 mg/ml.

24. A dermally deliverable pharmaceutical composition comprising a therapeutic agent solubilized in a solubilizing amount of a pharmaceutically acceptable carrier that comprises a low molecular weight monohydric alcohol, wherein the therapeutic agent comprises at least one selective COX-2 inhibitory drug or prodrug thereof and is present at a concentration in the composition of about 12.5 to about 400 mg/ml.

25. The composition of claim 24 wherein substantially all of the therapeutic agent present is in solubilized form.

26. The composition of claim 24 wherein the therapeutic agent comprises at least one compound having the formula 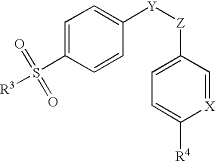
where R³ is a methyl, amino or imide group, R⁴ is hydrogen or a C₁₋₄ alkyl or alkoxy group, X is N or CR⁵ where R⁵ is hydrogen or halogen, and Y and Z are independently carbon or nitrogen atoms defining adjacent atoms of a five- to six-membered ring that is unsubstituted or substituted at one or more positions with oxo, halo, methyl or halomethyl groups; or an isomer, tautomer, pharmaceutically-acceptable salt or prodrug thereof.

27. The composition of claim 24 wherein the at least one selective COX-2 inhibitory drug or prodrug is selected from the group consisting of celecoxib, deracoxib, valdecoxib, parecoxib, rofecoxib, etoricoxib, 2-(3,5-difluorophenyl)-3-[4-(methylsulfonyl)phenyl]-2-cyclopenten-1-one, (S)-6,8-dichloro-2-(trifluoromethyl)-2H-1-benzopyran-3-carboxylic acid, 2-(3,4-difluorophenyl)-4-(3-hydroxy-3-methyl-1-butoxy)-5-[4-(methylsulfonyl)phenyl]-3-(2H)-pyridazinone, 5-methyl-2-(2′-chloro-6′-fluoroanilino)phenylacetic acid and salts thereof.

28. The composition of claim 24 wherein the at least one selective COX-2 inhibitory drug or prodrug is selected from the group consisting of celecoxib, valdecoxib, parecoxib and its salts, rofecoxib and etoricoxib.

29. The composition of claim 24 wherein the at least one selective COX-2 inhibitory drug or prodrug is valdecoxib or a prodrug thereof.

30. The composition of claim 24 wherein the at least one selective COX-2 inhibitory drug or prodrug is parecoxib or a salt thereof.

31. The composition of claim 24 wherein the monohydric alcohol is a C₂₋₆ monohydric alcohol.

32. The composition of claim 31 wherein the C₂₋₆ monohydric alcohol is selected from the group consisting of ethanol, isopropanol, n-butanol and diethylene glycol monoethyl ether.

33. The composition of claim 24 that is in a liquid or semi-solid dosage form.

34. The composition of claim 33 in a dosage form selected from the group consisting of creams, pastes, gels, ointments, lotions and aerosols.

35. The composition of claim 24 exhibiting a skin permeation rate of the therapeutic agent not less than about 10 μg/cm².day.

36. The composition of claim 24 exhibiting a skin permeation rate of the therapeutic agent not less than about 25 μg/cm².day.

37. The composition of claim 24, further comprising at least one skin permeation enhancer.

38. The composition of claim 37 wherein the at least one skin permeation enhancer is selected from the group consisting of terpenes, terpenoids, fatty alcohols and derivatives thereof, fatty acids and alkyl and glyceryl esters thereof, fatty acid esters of glycolic acid and its salts, lactate esters of fatty alcohols, laurocapram and derivatives thereof, dimethylsulfoxide, n-decyl methylsulfoxide, salicylic acid and alkyl esters thereof, N,N-dimethylacetamide, dimethylformamide, N,N-dimethyltoluamide, 2-pyrrolidinone and N-alkyl derivatives thereof, 2-nonyl-1,3-dioxolane, eucalyptol, sorbitan esters and sunscreens.

39. The composition of claim 37 wherein the at least one skin permeation enhancer is selected from the group consisting of oleyl alcohol, methyl salicylate, NMP, thymol, menthol, carvone, carveol, citral, dihydrocarveol, dihydrocarvone, neomenthol, isopulegol, 4-terpinenol, menthone, pulegol, camphor, geraniol, α-terpineol, linalool, carvacrol, trans-anethole, isomers thereof and racemic mixtures thereof.

40. The composition of claim 37 that comprises a fatty alcohol and a terpene or terpenoid as skin permeation enhancers.

41. The composition of claim 37 that comprises oleyl alcohol and thymol as skin permeation enhancers.

42. The composition of claim 37 wherein the at least one skin permeation enhancer is selected from the group consisting of oleic acid, isopropyl laurate, isopropyl myristate, methyl oleate, glyceryl monolaurate, glyceryl monooleate, glyceryl dilaurate, glyceryl dioleate, lauroyl glycolate, caproyl glycolate, cocoyl glycolate, isostearoyl glycolate, sodium lauroyl glycolate, tromethamine lauroyl glycolate, lauryl lactate, myristyl lactate and oleyl lactate.

43. The composition of claim 37 wherein the at least one skin permeation enhancer is glyceryl monolaurate.

44. The composition of claim 37 wherein the at least one skin permeation enhancer is a compound of formula 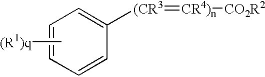
where R¹ groups are independently hydrogen, lower alkyl, lower alkoxy, halogen, hydroxyl or NR⁵R⁶ groups in which R⁵ and R⁶ are independently hydrogen or lower alkyl groups or R⁵ and R⁶ together with the nitrogen atom to which they are attached form a 5- or 6-membered heterocyclic ring; R² is a C₅₋₁₈ linear, branched or cyclic alkyl group; R³ is a hydrogen or phenyl group; R⁴ is a hydrogen or cyano group; n is 0 or 1; and q is 1 or 2.

45. The composition of claim 37 wherein the at least one skin permeation enhancer is selected from the group consisting of C₅₋₁₈ alkyl esters of p-aminobenzoic acid (PABA), p-dimethylaminobenzoic acid, 2-aminobenzoic acid, cinnamic acid, p-methoxycinnamic acid, salicylic acid and 2-cyano-3,3-diphenylacrylic acid.

46. A dermally deliverable pharmaceutical composition comprising a therapeutic agent solubilized in a solubilizing amount of a pharmaceutically acceptable carrier that comprises a low molecular weight monohydric alcohol, wherein the therapeutic agent comprises valdecoxib and/or a prodrug thereof and is present at a concentration in the composition of about 0.5 to about 400 mg/ml.

47. The composition of claim 46 wherein the therapeutic agent comprises parecoxib or a salt thereof.

48. The composition of claim 46 wherein the monohydric alcohol is a C₂₋₆ monohydric alcohol.

49. The composition of claim 48 wherein the C₂₋₆ monohydric alcohol is selected from the group consisting of ethanol, isopropanol, n-butanol and diethylene glycol monoethyl ether.

50. The composition of claim 46 that is in a liquid or semi-solid dosage form.

51. The composition of claim 50 in a dosage form selected from the group consisting of creams, pastes, gels, ointments, lotions and aerosols.

52. The composition of claim 46, further comprising at least one skin permeation enhancer.

53. The composition of claim 52 wherein the at least one skin permeation enhancer is selected from the group consisting of terpenes, terpenoids, fatty alcohols and derivatives thereof, fatty acids and alkyl and glyceryl esters thereof, fatty acid esters of glycolic acid and its salts, lactate esters of fatty alcohols, laurocapram and derivatives thereof, dimethylsulfoxide, n-decyl methylsulfoxide, salicylic acid and alkyl esters thereof, N,N-dimethylacetamide, dimethylformamide, N,N-dimethyltoluamide, 2-pyrrolidinone and N-alkyl derivatives thereof, 2-nonyl-1,3-dioxolane, eucalyptol, sorbitan esters and sunscreens.

54. The composition of claim 52 wherein the at least one skin permeation enhancer is selected from the group consisting of oleyl alcohol, methyl salicylate, NMP, thymol, menthol, carvone, carveol, citral, dihydrocarveol, dihydrocarvone, neomenthol, isopulegol, 4-terpinenol, menthone, pulegol, camphor, geraniol, α-terpineol, linalool, carvacrol, trans-anethole, isomers thereof and racemic mixtures thereof.

55. The composition of claim 52 that comprises a fatty alcohol and a terpene or terpenoid as skin permeation enhancers.

56. The composition of claim 52 that comprises oleyl alcohol and thymol as skin permeation enhancers.

57. The composition of claim 52 wherein the at least one skin permeation enhancer is selected from the group consisting of oleic acid, isopropyl laurate, isopropyl myristate, methyl oleate, glyceryl monolaurate, glyceryl monooleate, glyceryl dilaurate, glyceryl dioleate, lauroyl glycolate, caproyl glycolate, cocoyl glycolate, isostearoyl glycolate, sodium lauroyl glycolate, tromethamine lauroyl glycolate, lauryl lactate, myristyl lactate and oleyl lactate.

58. The composition of claim 52 wherein the at least one skin permeation enhancer is glyceryl monolaurate.

59. The composition of claim 52 wherein the at least one skin permeation enhancer is a compound of formula 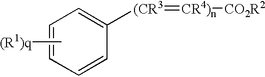
where R¹ groups are independently hydrogen, lower alkyl, lower alkoxy, halogen, hydroxyl or NR⁵R⁶ groups in which R⁵ and R⁶ are independently hydrogen or lower alkyl groups or R⁵ and R⁶ together with the nitrogen atom to which they are attached form a 5- or 6-membered heterocyclic ring; R is a C₅₋₁₈ linear, branched or cyclic alkyl group; R³ is a hydrogen or phenyl group; R⁴ is a hydrogen or cyano group; n is 0 or 1; and q is 1 or 2.

60. The composition of claim 52 wherein the at least one skin permeation enhancer is selected from the group consisting of C₅₋₁₈ alkyl esters of p-aminobenzoic acid (PABA), p-dimethylaminobenzoic acid, 2-aminobenzoic acid, cinnamic acid, p-methoxycinnamic acid, salicylic acid and 2-cyano-3,3-diphenylacrylic acid.

61. A dermally deliverable pharmaceutical composition in a form of a paste, ointment, gel or cream comprising at least one selective COX-2 inhibitory drug or prodrug in a total amount of 1.25% to 10%, at least one solvent in a total amount of 50% to 97%, at least one skin permeation enhancer in a total amount of 2% to 20% and at least one thickening agent in a total amount of 1% to 5%, by weight.

62. The composition of claim 61 wherein the at least one skin permeation enhancer is selected from the group consisting of terpenes, terpenoids, fatty alcohols and derivatives thereof, fatty acids and alkyl and glyceryl esters thereof, fatty acid esters of glycolic acid and its salts, lactate esters of fatty alcohols, laurocapram and derivatives thereof, dimethylsulfoxide, n-decyl methylsulfoxide, salicylic acid and alkyl esters thereof, N,N-dimethylacetamide, dimethylformamide, N,N-dimethyltoluamide, 2-pyrrolidinone and N-alkyl derivatives thereof, 2-nonyl-1,3-dioxolane, eucalyptol, sorbitan esters and sunscreens.

63. The composition of claim 61 wherein the at least one skin permeation enhancer is selected from the group consisting of oleyl alcohol, methyl salicylate, NMP, thymol, menthol, carvone, carveol, citral, dihydrocarveol, dihydrocarvone, neomenthol, isopulegol, 4-terpinenol, menthone, pulegol, camphor, geraniol, α-terpineol, linalool, carvacrol, trans-anethole, isomers thereof and racemic mixtures thereof.

64. The composition of claim 61 that comprises a fatty alcohol and a terpene or terpenoid as skin permeation enhancers.

65. The composition of claim 61 that comprises oleyl alcohol and thymol as skin permeation enhancers.

66. The composition of claim 61 wherein the at least one skin permeation enhancer is selected from the group consisting of oleic acid, isopropyl laurate, isopropyl myristate, methyl oleate, glyceryl monolaurate, glyceryl monooleate, glyceryl dilaurate, glyceryl dioleate, lauroyl glycolate, caproyl glycolate, cocoyl glycolate, isostearoyl glycolate, sodium lauroyl glycolate, tromethamine lauroyl glycolate, lauryl lactate, myristyl lactate and oleyl lactate.

67. The composition of claim 61 wherein the at least one skin permeation enhancer is glyceryl monolaurate.

68. The composition of claim 61 wherein the at least one skin permeation enhancer is a compound of formula 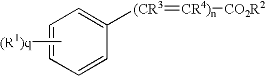
where R¹ groups are independently hydrogen, lower alkyl, lower alkoxy, halogen, hydroxyl or NR⁵R⁶ groups in which R⁵ and R⁶ are independently hydrogen or lower alkyl groups or R⁵ and R⁶together with the nitrogen atom to which they are attached form a 5- or 6-membered heterocyclic ring; R² is a C₅₋₁₈ linear, branched or cyclic alkyl group; R³ is a hydrogen or phenyl group; R⁴ is a hydrogen or cyano group; n is 0 or 1; and q is 1 or 2.

69. The composition of claim 61 wherein the at least one skin permeation enhancer is selected from the group consisting of C₅₋₁₈ alkyl esters of p-aminobenzoic acid (PABA), p-dimethylaminobenzoic acid, 2-aminobenzoic acid, cinnamic acid, p-methoxycinnamic acid, salicylic acid and 2-cyano-3,3-diphenylacrylic acid.

70. A dermally deliverable pharmaceutical composition in a form of a cream, paste, gel, ointment, lotion or aerosol comprising at least one selective COX-2 inhibitory drug or prodrug and a sunscreen.

71. The composition of claim 70 wherein the sunscreen is octyl p-dimethylaminobenzoate and is present in an amount of 1% to 10% by weight.

72. A method of effecting targeted delivery of a selective COX-2 inhibitory drug to a site of pain and/or inflammation in a subject, the method comprising topically administering the composition of claim 1 to skin of the subject.

73. The method of claim 72 wherein the composition is administered to skin at a locus overlying or adjacent to the site of pain and/or inflammation.

74. The method of claim 72 wherein the site of pain and/or inflammation is in an epidermal, dermal, subcutaneous, muscular or articular tissue.

75. A method of effecting targeted delivery of a selective COX-2 inhibitory drug to a site of pain and/or inflammation in a subject, the method comprising topically administering the composition of claim 24 to skin of the subject.

76. The method of claim 75 wherein the composition is administered to skin at a locus overlying or adjacent to the site of pain and/or inflammation.

77. The method of claim 75 wherein the site of pain and/or inflammation is in an epidermal, dermal, subcutaneous, muscular or articular tissue.

78. A method of effecting targeted delivery of a selective COX-2 inhibitory drug to a site of pain and/or inflammation in a subject, the method comprising topically administering the composition of claim 46 to skin of the subject.

79. The method of claim 78 wherein the composition is administered to skin at a locus overlying or adjacent to the site of pain and/or inflammation.

80. The method of claim 78 wherein the site of pain and/or inflammation is in an epidermal, dermal, subcutaneous, muscular or articular tissue.

81. A method of effecting targeted delivery of a selective COX-2 inhibitory drug to a site of pain and/or inflammation in a subject, the method comprising topically administering the composition of claim 61 to skin of the subject.

82. The method of claim 81 wherein the composition is administered to a skin surface at a locus overlying or adjacent to the site of pain and/or inflammation.

83. The method of claim 81 wherein the site of pain and/or inflammation is in an epidermal, dermal, subcutaneous, muscular or articular tissue.

84. A method of effecting targeted delivery of a selective COX-2 inhibitory drug to a site of pain and/or inflammation in a subject, the method comprising topically administering the composition of claim 70 to skin of the subject.

85. The method of claim 84 wherein the composition is administered to a skin surface at a locus overlying or adjacent to the site of pain and/or inflammation.

86. The method of claim 84 wherein the site of pain and/or inflammation is in an epidermal, dermal, subcutaneous, muscular or articular tissue.

87. A method of effecting systemic treatment of a subject having a COX-2 mediated disorder, the method comprising transdermally administering the composition of claim 1.

88. The method of claim 87 wherein the composition is contacted with an area of skin of the subject not greater than about 400 cm².

89. A method of effecting systemic treatment of a subject having a COX-2 mediated disorder, the method comprising transdermally administering the composition of claim 24.

90. The method of claim 89 wherein the composition is contacted with an area of skin of the subject not greater than about 400 cm².

91. A method of effecting systemic treatment of a subject having a COX-2 mediated disorder, the method comprising transdermally administering the composition of claim 46.

92. The method of claim 91 wherein the composition is contacted with an area of skin of the subject not greater than about 400 cm².

93. A method of effecting systemic treatment of a subject having a COX-2 mediated disorder, the method comprising transdermally administering the composition of claim 61.

94. The method of claim 93 wherein the composition is contacted with an area of skin of the subject not greater than about 400 cm².

95. A method of effecting systemic treatment of a subject having a COX-2 mediated disorder, the method comprising transdermally administering the composition of claim 70.

96. The method of claim 95 wherein the composition is contacted with an area of skin of the subject not greater than about 400 cm².
